(19) United States
(12) Patent Application Publication
(10) Pub. No.: US 2024/0272181 A1
(43) Pub. Date: Aug. 15, 2024

(54) METHODS AND COMPOSITIONS FOR DETERMINATION NON-ALCOHOLIC FATTY LIVER DISEASE (NAFLD) AND NON-ALCOHOLIC STEATOHEPATITIS (NASH)

(71) Applicants: The Regents of the University of California, Oakland, CA (US); Virginia Commonwealth University, Richmond, VA (US)

(72) Inventors: Edward A. Dennis, La Jolla, CA (US); Oswald Quehenberger, San Diego, CA (US); Rohit Loomba, San Diego, CA (US); Arun J. Sanyal, Richmond, VA (US)

(21) Appl. No.: 17/279,433

(22) PCT Filed: Oct. 4, 2019
(86) PCT No.: PCT/US2019/054864

§ 371 Date: Mar. 24, 2024

Related U.S. Application Data
(60) Provisional application No. 62/742,018, filed on Oct. 5, 2018.

(51) Int. Cl. G01N 33/92; G01N 30/72

(52) U.S. Cl. CPC G01N 33/92; G01N 30/72; G01N 2570/00; G01N 2800/085

(57) ABSTRACT

The disclosure provides methods for identifying non-alcoholic fatty liver disease and non-alcoholic steatohepatitis in a subject.

### CROSS REFERENCE TO RELATED APPLICATIONS

This application claims priority of U.S. Provisional Appl. No. 62/742,018, filed Oct. 5, 2018, the disclosures of which are incorporated herein by reference.

### STATEMENT OF GOVERNMENT SUPPORT

This invention was made with Government support under Grant No. DK105961 awarded by the National Institutes of Health. The Government has certain rights in the invention.

### FIELD OF THE INVENTION

The invention relates in general to materials and methods to quantitate markers to determine fatty liver disease.

### BACKGROUND

Fatty liver disease (or steatohepatis) is often associated with excessive alcohol intake or obesity, but also has other causes such as metabolic deficiencies including insulin resistance and diabetes. Fatty liver results from triglyceride fat accumulation in vacuoles of the liver cells resulting in decreased liver function, and possibly leading to cirrhosis or hepatic cancer.

Non-alcoholic fatty liver disease (NAFLD) represents a spectrum of disease occurring in the absence of alcohol abuse.

There is a clinical need for a simple test to identify individuals with nonalcoholic fatty liver disease (NAFLD) in the population as well as those with nonalcoholic steatohepatitis (NASH).

### SUMMARY

The disclosure provides a method of identifying nonalcoholic fatty liver disease (NAFLD) in a subject, comprising (a) obtaining a biological sample from the subject; (b) measuring the level of a plurality of bioactive lipids selected from the group consisting of at least dhk-PGD2, 5-HETE and ceramide P-d18: 1/20:5, and optionally one or more additional compounds selected from the group consisting of CER P-d18:1/18:0, SM 36:3, LPE 18:1, LPC 0-18:0, SM 34:3, PC 42:10, PC O—, LPC 18:2, PC 42:9, PC 0-42:2, and PC 40:0; and (c) comparing the levels of dhk-PGD2, 5-HETE and ceramide P-d18: 1/20:5 in the biological sample obtained from the subject to a control sample, wherein a difference in the levels is indicative of NAFLD. In one embodiment, the method comprises measuring at least dhk-PGD2, 5-HETE, ceramide P-d18: 1/20:5 and LPE 18:1. In another embodiment, the method comprises measuring at least dhk-PGD2, 5-HETE, ceramide P-d18:1/20:5, LPE 18:1 and SM 34:3. In yet another embodiment, the method comprises measuring at least dhk-PGD2, 5-HETE, ceramide P-d18:1/20:5, LPE 18:1, SM 34:3 and PC 43:9, PC 0-42:2. In still another embodiment, the method comprises measuring at least dhk-PGD2, 5-HETE, ceramide P-d18: 1/20:5, ceramide P-d18:1/18:0, and LPE 18:1. In yet another embodiment, the method comprises measuring at least dhk-PGD2, 5-HETE, ceramide P-d18:1/20:5, ceramide P-d18:1/18: 0, LPE 18:1, and SM 36:3. In still yet another embodiment, the method comprises measuring at least dhk-PGD2, 5-HETE, ceramide P-d18:1/20:5, ceramide P-d18:1/18:0, LPE 18:1, SM 36:3, and LPC O 18:0. In yet another embodiment, the method comprises measuring at least dhk-PGD2, 5-HETE, ceramide P-d18: 1/20:5, ceramide P-d18:1/18:0, LPE 18:1, SM 36:3, LPC O 18:0, and LPC 18:2. In yet another or further embodiment of any of the foregoing embodiments, the method comprises determining the area under receiver operating characteristic curve (AUROC) based upon a ratio of the levels of the bioactive lipids matched with deuterated internal standards of the same bioactive lipids. In yet another or further embodiment of any of the foregoing embodiments, the biological sample is selected from the group consisting of blood, blood plasma and blood serum. In yet another or further embodiment of any of the foregoing embodiments, the plurality of bioactive lipids are measured by liquid chromatography mass spectrometry. In yet another or further embodiment of any of the foregoing embodiments, the plurality of bioactive lipids are measured by gas chromatography mass spectrometry. In yet another or further embodiment of any of the foregoing embodiments, the method further comprises determining whether a subject with NAFLD has NASH by measuring a second set of bioactive lipids, selected from the group consisting of at least 14, 15-diHETrE, LPC 0-18:0 and PC 34:4, and optionally one or more additional compounds selected from the group consisting of LPC 20:5, PE 38:0, PE 0-40:7, PC 36:5, PC 40:8, PC 0-40:1 and PC 0-34:4 and wherein if there is a difference in the second set of bioactive lipids compared to a control or a control-NALFD level the levels are indicative of NASH.

The disclosure also provides a method of identifying nonalcoholic steatohepatitis (NASH) in a subject, comprising (a) obtaining a biological sample from the subject; (b) measuring the level of a plurality of bioactive lipids selected from the group consisting of at least 14, 15-diHETrE, LPC 0-18:0 and PC 34:4, and optionally one or more additional compounds selected from the group consisting of LPC 20:5, PE 38:0, PE 0-40:7, PC 36:5, PC 40:8, PC O-40:1 and PC 0-34:4; and (c) comparing the levels of at least 14, 15-diHETrE, LPC 0-18:0 and PC 34:4 in the biological sample obtained from the subject to a control sample, wherein a difference in the levels is indicative of NASH. In another embodiment, the method comprise measuring at least 14,15-diHETrE, LPC 0-18:0, PC 34:4 and PE 38:0, PE O-40:7. In yet another embodiment, the method comprises measuring at least 14,15-diHETrE, LPC 0-18:0, PC 34:4, and LPC 20:5. In still another embodiment, the method comprises measuring at least at least 14,15-diHETrE, LPC 0-18:0, PC 34:4, and PC 36:5. In still yet another embodiment, the method comprises measuring at least at least 14, 15-diHETrE, LPC 0-18:0, PC 34:4, and PC 40:8, PC 0-40:1. In yet another or further embodiment of any of the foregoing embodiments, the method comprises determining the area under receiver operating characteristic curve (AUROC) based upon a ratio of the levels of the bioactive lipids matched with deuterated internal standards of the same bioactive lipids. In yet another or further embodiment of any of the foregoing embodiments, the biological sample is selected from the group consisting of blood, blood plasma and blood serum. In yet another or further embodiment of any of the foregoing embodiments, the plurality of bioactive lipids are measured by liquid chromatography mass spectrometry. In yet another or further embodiment of any of the foregoing embodiments, the plurality of bioactive lipids are measured by gas chromatography mass spectrometry.

The disclosure also provides use of the levels of bioactive lipids selected from the group consisting of at least dhk-PGD2, 5-HETE and ceramide P-d18:1/20:5, and optionally one or more additional compounds selected from the group consisting of CER P-d18:1/18:0, SM 36:3, LPE 18:1, LPC 0-18:0, SM 34:3, PC 42:10, PC O—, LPC 18:2, PC 42:9, PC 0-42:2, and PC 40:0 obtained from a biological sample for producing a diagnosticum for the in vitro identification NAFLD.

The disclosure also provides use of the levels of bioactive lipids selected from the group consisting of at least 14, 15-diHETrE, LPC 0-18:0 and PC 34:4, and optionally one or more additional compounds selected from the group consisting of LPC 20:5, PE 38:0, PE 0-40:7, PC 36:5, PC 40:8, PC 0-40:1 and PC 0-34:4obtained from a biological sample for producing a diagnosticum for the in vitro differentiation of nonalcoholic steatohepatitis (NASH) from nonalcoholic fatty liver (NAFLD).

### BRIEF DESCRIPTION OF THE DRAWINGS

FIG. 1 shows a table of the top 20 lipids useful to discriminate any NASH from NAFLD.

FIG. 2A-B shows positive and negative predictive values (PPV and NPV) at varying prevalence using the final model fixed at 95% (A) and 97.5% (B) specificity.

FIG. 3A-B shows AIC values among the top 20 lipids with the lowest AIC.

FIG. 4 shows cross-validated Area under ROC curve of the final model.

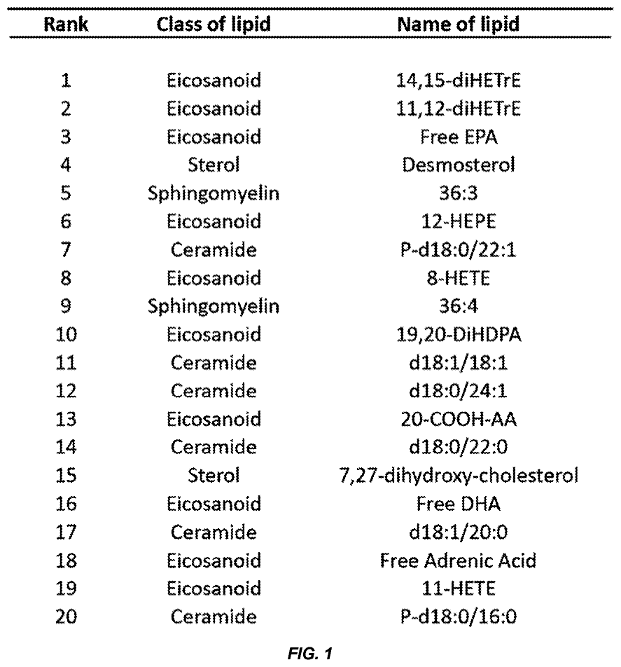

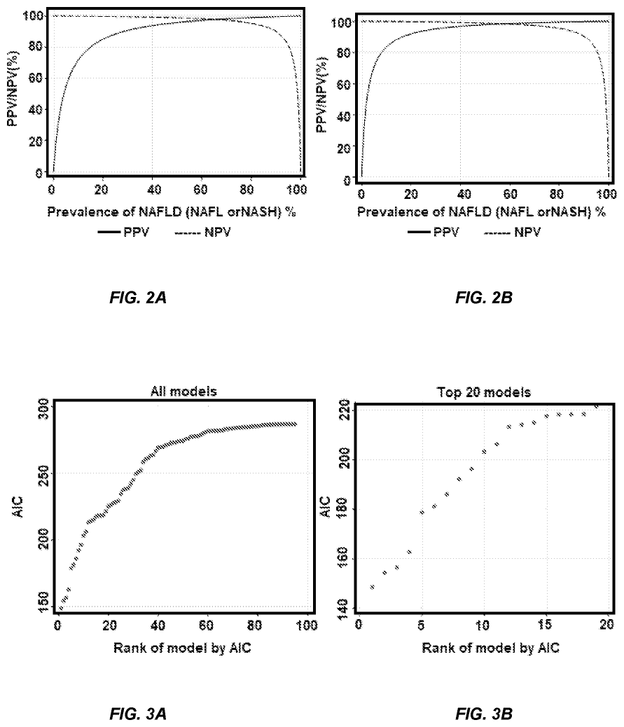

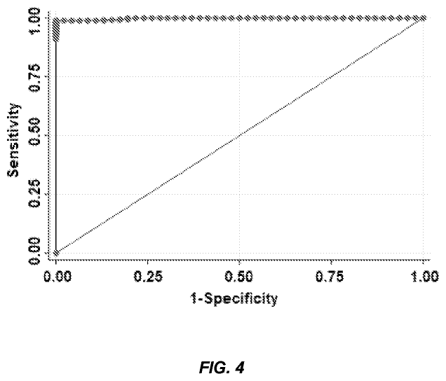

### DETAILED DESCRIPTION

As used herein and in the appended claims, the singular forms “a,” “an,” and “the” include plural referents unless the context clearly dictates otherwise. Thus, for example, reference to “an eicosanoid” includes a plurality of such eicosanoids and reference to “a subject” includes reference to one or more subjects and so forth.

Also, the use of “or” means “and/or” unless stated otherwise. Similarly, “comprise,” “comprises,” “comprising” “include,” “includes,” and “including” are interchangeable and not intended to be limiting.

It is to be further understood that where descriptions of various embodiments use the term “comprising,” those skilled in the art would understand that in some specific instances, an embodiment can be alternatively described using language “consisting essentially of” or “consisting of.”

Unless defined otherwise, all technical and scientific terms used herein have the same meaning as commonly understood to one of ordinary skill in the art to which this disclosure belongs. Although methods and materials similar or equivalent to those described herein can be used in the practice of the disclosed methods and compositions, the exemplary methods, devices and materials are described herein.

The publications discussed above and throughout the text are provided solely for their disclosure prior to the filing date of the present application. Nothing herein is to be construed as an admission that the inventors are not entitled to antedate such disclosure by virtue of prior disclosure.

“Biomarker” means a compound that is differentially present (i.e., increased or decreased) in a biological sample from a subject or a group of subjects having a first phenotype (e.g., having a disease) as compared to a biological sample from a subject or group of subjects having a second phenotype (e.g., not having the disease). A biomarker may be differentially present at any level, but is generally present at a level that is increased by at least 5%, by at least 10%, by at least 158, by at least 20%, by at least 25%, by at least 30%, by at least 35%, by at least 40%, by at least 45%, by at least 50%, by at least 55%, by at least 60%, by at least 65%, by at least 708, by at least 75%, by at least 80%, by at least 85%, by at least 90%, by at least 95%, by at least 100%, by at least 110%, by at least 120%, by at least 130%, by at least 140%, by at least 150%, or more; or is generally present at a level that is decreased by at least 58, by at least 108, by at least 15%, by at least 20%, by at least 25%, by at least 30%, by at least 358, by at least 40%, by at least 45%, by at least 50%, by at least 55%, by at least 60%, by at least 65%, by at least 708, by at least 758, by at least 80%, by at least 85%, by at least 908, by at least 958, or by 100% (i.e., absent). A biomarker is preferably differentially present at a level that is statistically significant.

As used herein, “biomarker level” and “level” refer to a measurement that is made using any analytical method for detecting the biomarker in a biological sample and that indicates the presence, absence, absolute amount or concentration, relative amount or concentration, titer, a level, an expression level, a ratio of measured levels, or the like, of, for, or corresponding to the biomarker in the biological sample. The exact nature of the “level” depends on the specific design and components of the particular analytical method employed to detect the biomarker.

As used herein, “detecting” or “determining” with respect to a biomarker level includes the use of both the instrument used to observe and record a signal corresponding to a biomarker level and the material (s) required to generate that signal. In various embodiments, the level is detected using any suitable method, including fluorescence, chemiluminescence, surface plasmon resonance, surface acoustic waves, mass spectrometry, infrared spectroscopy, Raman spectroscopy, atomic force microscopy, scanning tunneling microscopy, electrochemical detection methods, nuclear magnetic resonance, quantum dots, and the like.

“Diagnose”, “diagnosing”, “diagnosis”, and variations thereof refer to the detection, determination, or recognition of a health status or condition of an individual on the basis of one or more signs, symptoms, data, or other information pertaining to that individual. The health status of an individual can be diagnosed as healthy/normal (i.e., a diagnosis of the absence of a disease or condition) or diagnosed as ill/abnormal (i.e., a diagnosis of the presence, or an assessment of the characteristics, of a disease or condition). The terms “diagnose”, “diagnosing”, “diagnosis”, etc., encompass, with respect to a particular disease or condition, the initial detection of the disease; the characterization or classification of the disease; the detection of the progression, remission, or recurrence of the disease; and the detection of disease response after the administration of a treatment or therapy to the individual. The diagnosis of NAFLD includes distinguishing individuals who have NAFLD from individuals who do not. The diagnosis of NASH includes distinguishing individuals who have NASH from individuals who have steatosis in the liver, but not NASH, and from individuals with no liver disease.

A “reference level” or “reference sample level” of a biomarker means a level of the biomarker that is indicative of a particular disease state, phenotype, or predisposition to developing a particular disease state or phenotype, or lack thereof, as well as combinations of disease states, phenotypes, or predisposition to developing a particular disease state or phenotype, or lack thereof. A “positive” reference level of a biomarker means a level that is indicative of a particular disease state or phenotype. A “negative” reference level of a biomarker means a level that is indicative of a lack of a particular disease state or phenotype. A “reference level” of a biomarker may be an absolute or relative amount or concentration of the biomarker, a presence or absence of the biomarker, a range of amount or concentration of the biomarker, a minimum and/or maximum amount or concentration of the biomarker, a mean amount or concentration of the biomarker, and/or a median amount or concentration of the biomarker; and, in addition, “reference levels” of combinations of biomarkers may also be ratios of absolute or relative amounts or concentrations of two or more biomarkers with respect to each other. Appropriate positive and negative reference levels of biomarkers for a particular disease state, phenotype, or lack thereof may be determined by measuring levels of desired biomarkers in one or more appropriate subjects, and such reference levels may be tailored to specific populations of subjects (e.g., a reference level may be age-matched or gender-matched so that comparisons may be made between biomarker levels in samples from subjects of a certain age or gender and reference levels for a particular disease state, phenotype, or lack thereof in a certain age or gender group). Such reference levels may also be tailored to specific techniques that are used to measure levels of biomarkers in biological samples (e.g., LC-MS, GC-MS, etc.), where the levels of biomarkers may differ based on the specific technique that is used. A “control level” of a target molecule refers to the level of the target molecule in the same sample type from an individual that does not have the disease or condition, or from an individual that is not suspected of having the disease or condition. A “control level” of a target molecule need not be determined each time the present methods are carried out, and may be a previously determined level that is used as a reference or threshold to determine whether the level in a particular sample is higher or lower than a normal level. In some embodiments, a control level in a method described herein is the level that has been observed in one or more subjects (i.e., a population) without NAFLD. In some embodiments, a control level in a method described herein is the level that has been observed in one or more subjects with NAFLD, but not NASH. In some embodiments, a control level in a method described herein is the average or mean level, optionally plus or minus a statistical variation that has been observed in a plurality of normal subjects, or subjects with NAFLD but not NASH.

Non-alcoholic fatty liver disease (NAFLD) represents a spectrum of disease occurring in the absence of alcohol abuse. It is characterized by the presence of steatosis (fat in the liver) and may represent a hepatic manifestation of the metabolic syndrome (including obesity, diabetes and hypertriglyceridemia). NAFLD is linked to insulin resistance, it causes liver disease in adults and children and may ultimately lead to cirrhosis (Skelly et al., J Hepatol., 35: 195-9, 2001; Chitturi et al., Hepatology, 35 (2): 373-9, 2002). The severity of NAFLD ranges from the relatively benign isolated predominantly macrovesicular steatosis (i.e., nonalcoholic fatty liver (NAFL)) to non-alcoholic steatohepatitis (NASH) (Angulo et al., J Gastroenterol Hepatol, 17 Suppl: S186-90, 2002). NASH is characterized by the histologic presence of steatosis, cytological ballooning, scattered inflammation and pericellular fibrosis (Contos et al., Adv Anat Pathol., 9:37-51, 2002). Hepatic fibrosis resulting from NASH may progress to cirrhosis of the liver or liver failure, and in some instances may lead to hepatocellular carcinoma.

The degree of insulin resistance (and hyperinsulinemia) correlates with the severity of NAFLD, being more pronounced in patients with NASH than with simple fatty liver (Sanyal et al., Gastroenterology, 120 (5): 1183-92, 2001). As a result, insulin-mediated suppression of lipolysis occurs and levels of circulating fatty acids increase. Two factors associated with NASH include insulin resistance and increased delivery of free fatty acids to the liver. Insulin blocks mitochondrial fatty acid oxidation. The increased generation of free fatty acids for hepatic re-esterification and oxidation results in accumulation of intrahepatic fat and increases the liver's vulnerability to secondary insults.

The prevalence of NAFLD in children is unknown because of the requirement of histologic analysis of liver in order to confirm the diagnosis (Schwimmer et al., Pediatrics, 118 (4): 1388-93, 2006). However, estimates of prevalence can be inferred from pediatric obesity data using hepatic ultra-sonongraphy and elevated serum transaminase levels and the knowledge that 85% of children with NAFLD are obese. Data from the National Health and Nutrition Examination Survey has revealed a threefold rise in the prevalence of childhood and adolescent obesity over the past 35 years; data from 2000 suggests that 14-168 children between 6-19 yrs age are obese with a BMI >95% (Fishbein et al., J Pediatr. Gastroenterol. Nutr., 36(1): 54-61, 2003), and also that fact that 85% of children with NAFLD are obese.

In patients with histologically proven NAFLD, serum hepatic aminotransferases, specifically alanine aminotransferase (ALT), levels are elevated from the upper limit of normal to 10 times this level (Schwimmer et al., J Pediatr., 143 (4): 500-5, 2003; Rashid et al., J Pediatr Gastroenterol Nutr., 30 (1): 48-53, 2000). The ratio of ALT/AST (aspartate aminotransferase) is >1 (range 1.5-1.7) which differs from alcoholic steatohepatitis where the ratio is generally <1. Other abnormal serologic tests that may be abnormally elevated in NASH include gamma-glutamyltransferase (gamma-GT) and fasting levels of plasma insulin, cholesterol and triglyceride.

The exact mechanism by which NAFLD develops into NASH remains unclear. Because insulin resistance is associated with both NAFLD and NASH, it is postulated that other additional factors are also required for NASH to arise. This is referred to as the “two-hit” hypothesis (Day CP. Best Pract. Res. Clin. Gastroenterol., 16 (5): 663-78, 2002) and involves, firstly, an accumulation of fat within the liver and, secondly, the presence of large amounts of free radicals with increased oxidative stress. Macrovesicular steatosis represents hepatic accumulation of triglycerides, and this in turn is due to an imbalance between the delivery and utilization of free fatty acids to the liver. During periods of increased calorie intake, triglyceride will accumulate and act as a reserve energy source. When dietary calories are insufficient, stored triglycerides (in adipose) undergo lipolysis and fatty acids are released into the circulation and are taken up by the liver. Oxidation of fatty acids will yield energy for utilization.

Bioactive lipids include a number of molecules whose concentrations or presence affect cellular function. Bioactive lipids, as used herein, include phospholipids, sphingolipids, lysophospholipids, ceramides, diacylglycerol, eicosanoids, steroid hormones and the like. Eicosanoids and related metabolites, sometimes referred to as oxylipins, are a group of structurally diverse metabolites that derive from the oxidation of polyunsaturated acids (PUFAs) including arachidonic acid (AA), linoleic acid, alpha and gamma linolenic acid, dihomo gamma linolenic acid, eicosapentaenoic acid and docosahexaenoic acid. They are locally acting bioactive signaling lipids that regulate a diverse set of homeostatic and inflammatory processes. Given the important regulatory functions in numerous physiological and pathophysiological states, the accurate measurement of eicosanoids and other oxylipins is of great clinical interest and lipidomics is now widely used to screen effectively for potential disease biomarkers.

The biosynthesis of eicosanoids and oxylipins involves the action of multiple enzymes organized into a complex and intertwined lipid-anabolic network. Generally, the enzymatic formation of eicosanoids requires free fatty acids as substrates; thus, the pathway is initiated by the hydrolysis of phospholipids (PLs) by phospholipase A₂ upon physiological stimuli. The hydrolyzed PUFAs are then processed by three enzyme systems: cyclooxygenases (COX), lipoxygenases (LOX), and cytochrome P450 enzymes (CYP450). Each of these enzyme systems produces unique collections of oxygenated metabolites that function as end-products or as intermediates for a cascade of downstream enzymes. The resulting eicosanoids exhibit diverse biological activities, half-lives and utilities in regulating many physiological processes in health and disease including the immune response, inflammation, and homeostasis. Additionally, non-enzymatic processes can produce oxidized PUFA metabolites via free radical reactions giving rise to isoprostanes and other oxidized fatty acids.

Eicosanoids act locally in an autocrine or paracrine fashion and signal by binding to G-protein-coupled receptors or act intracellularly via various peroxisome proliferator-activating receptors. For optimal biological activity, these mediators need to be present in their free, non-esterified form. However, a number of studies reported that a portion of eicosanoids are naturally esterified and can also be contained in cell membrane lipids, including PLs, in the form of esters. The role of esterified eicosanoids is not clear but they may be signaling molecules in their own right or serve as a cellular reservoir for the rapid release upon cell stimulation.

Two potential mechanisms for the formation of eicosanoids-containing PLs have been proposed: (i) direct oxidation of PUFAs on the intact PLs, and (ii) re-acylation of preformed free oxylipins into lysoPLs. Cyclooxygenases require free fatty acid as substrate and show little activity toward PUFAs in intact PLs. A number of subsequent studies support the concept that prostaglandins are first formed enzymatically and then incorporated into PLs by the sequential actions of long-chain acyl-CoA synthases and lysophospholipid acyltransferases. Additionally, preformed fatty acid epoxides, including the regioisomers of epoxyeicosatrienoic acid (EET), are effectively incorporated primarily into the phospholipid fraction of cellular lipids, presumably via CoA-dependent mechanisms.

In contrast, mammalian 12/15 lipoxygenase (LOX) can act directly on PLs to generate esterified HETE isomers including esterified 12-HETE and 15-HETE. Similarly, the endocannabinoid 2-arachidonylglycerol is a substrate for COX-2 and is metabolized to prostaglandin H2 glycerol ester as effectively as free AA. The final products derived from this direct PL oxygenation pathway include esterified prostaglandins (PGs) as well as 11-HETE and 15-HETE. PUFAs contained in PLs can also be oxidized by non-enzymatic reactions. Free radical peroxidation reactions observed under conditions of oxidative stress can freely proceed on intact PLs resulting in the formation of isoprostanes.

As described below and elsewhere herein LC-MS/MS protocols are described to demonstrate that plasma levels of oxylipins can be used as biomarkers to identify subjects having or at risk of having nonalcoholic fatty liver disease (NAFLD) as well as differentiate the progressive form of nonalcoholic fatty liver disease, termed nonalcoholic steatohepatitis (NASH), from the milder form termed nonalcoholic fatty liver (NAFL). In this method, a panel of oxylipins that, when used together, can discriminate controls from NAFLD and NASH from NAFL with a high degree of certainty.

The disclosure includes the measurements of bioactive lipids. In some embodiments, methods were used to measure the “free” oxylipins present in plasma, not those appearing after alkaline hydrolysis (see, Feldstein et al.). In other embodiment, the sum total of esterified and free oxylipins are used by treating the sample with alkali (e.g., KOH).

For example, eicosanoids and specifically PGs are sensitive to alkaline-induced degradation. Thus, experiments presented herein were performed to minimize degradation of lipid metabolites during alkaline treatment and to identify specific eicosanoids and related oxidized PUFAs that are released intact from esterified lipids and which can be quantitatively measured.

The eicosanoid biosynthetic pathway includes over 100 bioactive lipids and relevant enzymes organized into a complex and intertwined lipid-signaling network. Biosynthesis of polyunsaturated fatty acid (PUFA) derived lipid mediators is initiated via the hydrolysis of phospholipids by phospholipase A₂ (PLA₂) upon physiological stimuli. These PUFA including arachidonic acid (AA), dihomo-gamma-linolenic acid (DGLA), eicosapentaenoic acid (EPA), and docosahexaenoic acid (DHA) are then processed by three enzyme systems: lipoxygenases (LOX), cyclooxygenases (COX) and cytochrome P450s, producing three distinct lineages of oxidized lipid classes. These enzymes are all capable of converting free arachidonic acid and related PUFA to their specific metabolites and exhibit diverse potencies, half-lives and utilities in regulating inflammation and signaling. Additionally, non-enzymatic processes can result in oxidized PUFA metabolites including metabolites from the essential fatty acids linoleic (LA) and alpha-linolenic acid (ALA).

Eicosanoids, which are key regulatory molecules in metabolic syndromes and the progression of hepatic steatosis to steatohepatitis in nonalcoholic fatty liver disease (NAFLD), act either as anti-inflammatory agents or as pro-inflammatory agents. Convincing evidence for a causal role of lipid peroxidation in steatohepatitis has not been unequivocally established; however, a decade of research has strongly suggested that these processes occur and that oxidative-stress is associated with hepatic toxicity and injury. As discussed above, nonalcoholic fatty liver disease (NAFLD) encompasses a wide spectrum of histological cases associated with hepatic fat over-accumulation that range from nonalcoholic fatty liver (NAFL) to nonalcoholic steatohepatitis (NASH). It is distinguished from NAFL by evidence of cytological ballooning, inflammation, and higher degrees of scarring and fibrosis. Hence, NASH is a serious condition, and approximately 10-25% of inflicted patients eventually develop advanced liver disease, cirrhosis, and hepatocellular carcinoma.

Thus, it is important to differentiate NASH from NAFL. At the present time, the gold standard technique for the diagnosis of NASH is a liver biopsy examination, which is recognized as the only reliable method to evaluate the presence and extent of necro-inflammatory changes, presence of ballooning and fibrosis in liver. However, liver biopsy is an invasive procedure with possible serious complications and limitations. Reliable noninvasive methods are therefore needed to avoid the sampling risks. It is proposed that differences in plasma levels of free eicosanoids can distinguish NAFL from NASH based on studies of well-characterized patients with biopsy substantiated NAFL and NASH.

Alterations in lipid metabolism may give rise to hepatic steatosis due to increased lipogenesis, defective peroxisomal and mitochondrial B-oxidation, and/or a lower ability of the liver to export lipids resulting in changes in fatty acids and/or eicosanoids. Some studies have highlighted the role of triacylglycerol, membrane fatty acid composition, and very low density lipoprotein (VLDL) production in the development of NASH and associated metabolic syndromes.

Cyclooxygenase-2 (COX-2), a key enzyme in eicosanoid metabolism, is abundantly expressed in NASH, which promotes hepatocellular apoptosis in rats. Others have reported that oxidized lipid products of LA including 9-hydroxyoctadienoic acid (9-HODE), 13-HODE, 9-oxooctadienoic acid (9-oxoODE), and 13-oxoODE as well as of arachidonic acid 5-hydroxyeicosa-tetraenoic acid (5-HETE), 8-HETE, 11-HETE, and 15-HETE are linked to histological severity in nonalcoholic fatty liver disease.

Free fatty acids are cytotoxic; thus the majority of all fatty acids in mammalian systems are esterified to phospholipids and glycerolipids as well as other complex lipids. Similarly, oxygenated metabolites of fatty acids can exist either in their free form or esterified to complex lipids.

The disclosure provides methods, kits and compositions useful for differentiation NAFL from NASH or identifying stages in NAFLD. In addition, the disclosure provides methods of identifying subject having or at risk of having NALFD. Such methods will help in the early onset and treatment of disease. Moreover, the methods reduce biopsy risks associated with liver biopsies currently used in diagnosis. The methods and compositions comprise modified eicosanoids and PUFAs in the diagnosis. As such, the biomarkers are manipulated from their natural state by chemical modifications to provide a derived biomarker that is measured and quantitated. The amount of a specific biomarker can be compared to normal standard sample levels (i.e., those lacking any liver disease) or can be compared to levels obtained from a diseased population (e.g., populations with clinically diagnosed NASH or NAFL).

Levels of free eicosanoids and PUFA metabolites can be expressed as AUROC (Area under Receiver Operating Characteristic Curve). AUROC is determined by measuring levels of free eicosanoids and PUFA metabolites by stable isotope dilution. Briefly, identical amounts of deuterated internal standards are added to each sample and to all the primary standards used to generate standard curves. Levels of eicosanoids and PUFA metabolites are calculated by determining the ratios between endogenous metabolite and matching deuterated internal standards. Ratios are converted to absolute amounts by linear regression. Individual eicosanoid metabolites are assessed to identify differences between levels in control, NAFL and NAFLD using statistical analyses including chi-square test, t-test and AUROC.

The method of the disclosure comprises determining the level of one or more free eicosanoids and/or polyunsaturated fatty acid (PUFA) metabolites in a sample of a patient. As used herein, the term “sample” refers to any biological sample from a patient. Examples include, but are not limited to, saliva, hair, skin, tissue, sputum, blood, plasma, serum, vitreal, cerebrospinal fluid, urine, sperm and cells. In one embodiment, the sample is a plasma sample.

Lipids are extracted from the sample, as detailed further in the Examples. The identity and quantity of bioactive lipids, eicosanoids and/or PUFA metabolites in the extracted lipids is first determined and then compared to suitable controls (e.g., a sample indicative of a subject with no liver disease, a sample indicative of a subject with NAFLD and/or a sample indicative of a subject with NASH). The determination may be made by any suitable lipid assay technique, such as a high throughput technique including, but not limited to, spectrophotometric analysis (e.g., colorimetric sulfo-phospho-vanillin (SPV) assessment method of Cheng et al., Lipids, 46 (1): 95-103 (2011)). Other analytical methods suitable for detection and quantification of lipid content will be known to those in the art including, without limitation, ELISA, NMR, UV-Vis or gas-liquid chromatography, HPLC, UPLC and/or MS or RIA methods enzymatic based chromogenic methods. Lipid extraction may also be performed by various methods known to the art, including the conventional method for liquid samples described in Bligh and Dyer, Can. J. Biochem. Physiol., 37, 91 1 (1959).

The disclosure demonstrates that out of 216 (65, 536 combination) possible combinations of 16 lipids, 20 models were developed based upon a review of the bioactive lipids present in control and NALFD subject. Table A provides a list of suitable panels for use in the methods of the disclosure to identify subject having or at risk of having NAFLD.

TABLE A
Top best 20 models:

| | Lipids included in the model | | | | | | | | | |
|---|---|---|---|---|---|---|---|---|---|---|
| | (“x” indicates inclusion in the model) | | | | | | | | | |
| | Lipids sorted by the rank in the previous 1-lipid model | | | | | | | | | |
| Rank of | CER | SM | LPE | LPC | SM | PC | LPC | PC 42:9, | CER | dhk |
| model | P-d18:1/18:0 | 36:3 | 18:1 | O-18:0 | 34:3 | 42:10, PC O- | 18:2 | PC O-42:2 | P-d18:0/18:0 | PGD1 |
| 1 | | | x | | x | | | x | | x |
| 2 | x | x | x | x | | | x | | | x |
| 3 | | | x | | x | x | | | | x |
| 4 | | x | x | | | x | | | | x |
| 5 | x | | x | x | x | | x | | | |
| 6 | | x | x | | | | | x | | |
| 7 | | | x | | x | | | x | | |
| 8 | x | x | x | x | x | | x | | | |
| 9 | | | x | x | x | x | | | | |
| 10 | | x | | | x | x | | | | |
| 11 | | | x | | x | x | | | | |
| 12 | | x | x | | x | | | x | | |
| 13 | | x | | | | x | | | | |
| 14 | x | | x | x | x | | | | | |
| 15 | | x | x | x | x | | | x | | |
| 16 | x | | x | x | x | | | x | | |
| 17 | x | | x | x | x | x | | | | |
| 18 | | x | | x | x | | | x | | |
| 19 | | x | x | x | x | x | | | | |
| 20 | | x | x | | | | | x | | |

| | Lipids included in the model | | | | | | | | |
|---|---|---|---|---|---|---|---|---|---|
| | (“x” indicates inclusion in the model) | | | | | | | | |
| | Lipids sorted by the rank in the previous 1-lipid model | | | | | | No. of | | |
| Rank of | LPC | CER | 5,6- | CER | PC | 5- | included | | |
| model | O-16:0 | d18:1/24:1 | diHETrE | P-d18:1/20:5 | 40:0 | HETE | lipids | BIC | AUROC |
| 1 | | | | x | | x | 6 | 84.9 | 0.996 |
| 2 | | | | x | | x | 8 | 85.2 | 0.998 |
| 3 | | | | x | | x | 6 | 85.8 | 0.996 |
| 4 | | | | x | x | x | 7 | 86.3 | 0.997 |
| 5 | | | | x | | x | 8 | 86.4 | 0.998 |
| 6 | | | | x | x | x | 7 | 86.4 | 0.997 |
| 7 | | | | x | x | x | 7 | 86.8 | 0.997 |
| 8 | | | | x | | x | 9 | 86.9 | 0.998 |
| 9 | | | | x | | x | 7 | 86.9 | 0.997 |
| 10 | | | | x | x | x | 7 | 86.9 | 0.997 |
| 11 | | | | x | x | x | 7 | 87.1 | 0.997 |
| 12 | | | | x | | x | 7 | 87.1 | 0.996 |
| 13 | | | | x | x | x | 6 | 87.1 | 0.996 |
| 14 | | | | x | | x | 7 | 87.2 | 0.997 |
| 15 | | | | x | x | x | 8 | 87.2 | 0.998 |
| 16 | | | | x | | x | 8 | 87.3 | 0.998 |
| 17 | | | | x | | x | 8 | 87.4 | 0.998 |
| 18 | | | | x | x | x | 7 | 87.4 | 0.997 |
| 19 | | | | x | x | x | 8 | 87.5 | 0.998 |
| 20 | | | | x | | x | 6 | 87.5 | 0.996 |

Accordingly, in one method of the disclosure, the method comprises obtaining a sample from a subject (e.g., a plasma sample), extracting the bioactive lipids in the sample and determining the levels of at least dhk-PGD2, 5-HETE and ceramide P-d18: 1/20:5. In another embodiment, the method can include measuring at least dhk-PGD2, 5-HETE, ceramide P-d18: 1/20:5 and LPE 18:1. In another embodiment, the method can include measuring at least dhk-PGD2, 5-HETE, ceramide P-d18:1/20:5, LPE 18:1 and SM 34:3. In another embodiment, the method can include measuring at least dhk-PGD2, 5-HETE, ceramide P-d18: 1/20:5, LPE 18:1, SM 34:3 and PC 43:9, PC O-42:2. In another embodiment, the method can include measuring at least dhk-PGD2, 5-HETE, ceramide P-d18: 1/20:5, ceramide P-d18:1/18:0, and LPE 18:1. In yet another embodiment, the method can include measuring at least dhk-PGD2, 5-HETE, ceramide P-d18: 1/20: 5, ceramide P-d18:1/18:0, LPE 18:1, and SM 36:3. In still another embodiment, the method can include measuring at least dhk-PGD2, 5-HETE, ceramide P-d18:1/20:5, ceramide P-d18:1/18:0, LPE 18:1, SM 36:3, and LPC O 18:0. In yet another embodiment, the method can include measuring at least dhk-PGD2, 5-HETE, ceramide P-d18: 1/20: 5, ceramide P-d18:1/18:0, LPE 18:1, SM 36:3, LPC O 18:0, and LPC 18:2. It is to be understood that the disclosure contemplates measuring the 20 possible combinations of Table A. The measurements are compared to a control or reference level (e.g., levels associated with a subject lacking NAFLD or lacking NASH), wherein a statistically significant different in the markers is indicative of NAFLD or NASH (as the case may be). Moreover, it will be recognized that the reference level will be a reference level for the particular type of measurement used.

The disclosure provides a substantially non-invasive method of predicting or assessing the risk of progression of liver disease in a patient comprising obtaining a plasma sample from a subject and optionally treating the plasma sample with alcohol to dissolve free eicosanoids and free polyunsaturated fatty acid (fPUFA) to obtain free-dissolved eicosanoids and free-dissolved fPUFAs; purifying bioactive lipids including eicosanoids and PUFAs; measuring the level of bioactive lipids selected from the group consisting of at least dhk-PGD2, 5-HETE and ceramide P-d18: 1/20:5, and optionally one or more additional compounds selected from the group consisting of CER P-d18:1/18:0, SM 36:3, LPE 18:1, LPC 0-18:0, SM 34:3, PC 42:10, PC O—, LPC 18:2, PC 42:9, PC 0-42:2, and PC 40:0; determining the area under receiver operating characteristic curve (AUROC) based upon a ratio of the levels of the bioactive lipids matched with deuterated internal standards of the same metabolite. In another embodiment, the method can include measuring at least dhk-PGD2, 5-HETE, ceramide P-d18:1/20:5 and LPE 18:1. In another embodiment, the method can include measuring at least dhk-PGD2, 5-HETE, ceramide P-d18:1/20:5, LPE 18:1 and SM 34:3. In another embodiment, the method can include measuring at least dhk-PGD2, 5-HETE, ceramide P-d18:1/20:5, LPE 18:1, SM 34:3 and PC 43:9, PC 0-42:2. In another embodiment, the method can include measuring at least dhk-PGD2, 5-HETE, ceramide P-d18: 1/20:5, ceramide P-d18:1/18:0, and LPE 18:1. In yet another embodiment, the method can include measuring at least dhk-PGD2, 5-HETE, ceramide P-d18:1/20:5, ceramide P-d18:1/18:0, LPE 18:1, and SM 36:3. In still another embodiment, the method can include measuring at least dhk-PGD2, 5-HETE, ceramide P-d18: 1/20:5, ceramide P-d18:1/18:0, LPE 18:1, SM 36:3, and LPC O 18:0. In yet another embodiment, the method can include measuring at least dhk-PGD2, 5-HETE, ceramide P-d18: 1/20:5, ceramide P-d18:1/18:0, LPE 18:1, SM 36:3, LPC O 18:0, and LPC 18:2. In one embodiment, the liver disease is a nonalcoholic fatty liver disease (NAFLD). In another embodiment, the NAFLD is nonalcoholic steatohepatitis (NASH). In another or further embodiment, the AUROC is about at least 0.8, at least about 0.9, or at least about 0.99.

In another embodiment, the disclosure provides a substantially non-invasive method of predicting or assessing the risk of progression of liver disease in a patient diagnosed with liver disease comprising obtaining a plasma sample from a subject, spiking deuterated internal standards into each sample and primary standards used to generate a standard curve and optionally treating the plasma sample with alcohol to dissolve free eicosanoids and free polyunsaturated fatty acid (fPUFA) to obtain free-dissolved eicosanoids and free-dissolved fPUFAs; purifying bioactive lipids including eicosanoids and PUFAs; measuring the level of bioactive lipids selected from the group consisting of at least dhk-PGD2, 5-HETE and ceramide P-d18: 1/20:5, and optionally one or more additional compounds selected from the group consisting of CER P-d18:1/18:0, SM 36:3, LPE 18:1, LPC 0-18:0, SM 34:3, PC 42:10, PC O—, LPC 18:2, PC 42:9, PC 0-42:2, and PC 40:0; calculating the ratio between endogenous metabolite and matching deuterated internal standards, converting the ratios to absolute amounts by linear regression, determining the area under receiver operating characteristic curve (AUROC) based upon a ratio of the levels of the bioactive lipids matched with deuterated internal standards of the same metabolite. In another embodiment, the method can include measuring at least dhk-PGD2, 5-HETE, ceramide P-d18: 1/20:5 and LPE 18:1. In another embodiment, the method can include measuring at least dhk-PGD2, 5-HETE, ceramide P-d18: 1/20:5, LPE 18:1 and SM 34:3. In another embodiment, the method can include measuring at least dhk-PGD2, 5-HETE, ceramide P-d18:1/20:5, LPE 18:1, SM 34:3 and PC 43:9, PC 0-42:2. In another embodiment, the method can include measuring at least dhk-PGD2, 5-HETE, ceramide P-d18: 1/20:5, ceramide P-d18:1/18:0, and LPE 18:1. In yet another embodiment, the method can include measuring at least dhk-PGD2, 5-HETE, ceramide P-d18:1/20:5, ceramide P-d18: 1/18: 0, LPE 18:1, and SM 36:3. In still another embodiment, the method can include measuring at least dhk-PGD2, 5-HETE, ceramide P-d18:1/20:5, ceramide P-d18:1/18:0, LPE 18:1, SM 36:3, and LPC O 18:0. In yet another embodiment, the method can include measuring at least dhk-PGD2, 5-HETE, ceramide P-d18:1/20:5, ceramide P-d18:1/18:0, LPE 18:1, SM 36:3, LPC O 18:0, and LPC 18:2. In one embodiment, the liver disease is a nonalcoholic fatty liver disease (NAFLD). In another embodiment, the NAFLD is nonalcoholic steatohepatitis (NASH). In another or further embodiment, the AUROC is about at least 0.8, at least about 0.9, or at least about 0.99.

The disclosure demonstrates that out of 29 possible combinations of 9 lipids, 20 models were developed based upon a review of the bioactive lipids present in NASH vs. NALFD subject. Table B provides a list of suitable panels for use in the methods of the disclosure to identify subject having or at risk of having NASH.

TABLE B
Top best 20 models - results of best subset selections* (N = 304)

| | Lipids included in the model | | | | | | | | | | | |
|---|---|---|---|---|---|---|---|---|---|---|---|---|
| | “x” indicates inclusion in the model) | | | | | | | | | | | |
| | Lipids sorted by the rank in the previous 1-lipid model | | | | | | | | | No. of | | |
| Rank of | 14,15- | LPC | LPC | PE 38:0, | PC | PC | PC 40:8, | PC | 11,12- | included | | |
| model | diHETrE | 20:5 | O-18:0 | PE O-40:7 | 36:5 | 34:4 | PC O-40:1 | O-34:4 | diHETrE | lipids | BIC | AUROC |

| | | | | | | | | | | | |
|---|---|---|---|---|---|---|---|---|---|---|---|
| 1 | x | | x | | | x | | | 3 | 344.3 | 0.688 |
| 2 | x | | x | x | | | | | 3 | 345.0 | 0.694 |
| 3 | x | | x | | x | | | | 3 | 346.0 | 0.692 |
| 4 | x | | x | | | | | | 2 | 346.0 | 0.675 |
| 5 | x | x | | | | x | | | 3 | 346.9 | 0.684 |
| 6 | x | x | | | | | | | 2 | 346.9 | 0.674 |
| 7 | x | | x | x | | x | | | 4 | 347.1 | 0.699 |
| 8 | x | x | x | | | x | | | 4 | 347.9 | 0.692 |
| 9 | x | | x | | | | x | | 3 | 348.1 | 0.678 |
| 10 | x | | x | | x | x | | | 4 | 348.1 | 0.696 |
| 11 | | | x | x | | | | | 2 | 348.4 | 0.672 |
| 12 | x | x | x | | | | | | 3 | 349.0 | 0.684 |
| 13 | x | | x | x | x | | | | 4 | 349.0 | 0.697 |
| 14 | x | x | | | | | x | | 3 | 349.2 | 0.673 |
| 15 | x | | x | | | x | x | | 4 | 349.7 | 0.689 |
| 16 | x | x | | x | | | | | 3 | 349.7 | 0.674 |
| 17 | x | | x | | | x | | x | 4 | 349.8 | 0.690 |
| 18 | x | | x | | | | | x | 3 | 349.8 | 0.681 |
| 19 | x | x | x | x | | | | | 4 | 349.8 | 0.695 |
| 20 | | | x | | x | | | | 2 | 350.0 | 0.656 |

LPC = lyso-phosphatidylcholine, PC = phosphatidylcholine
*Among 2⁹ = 512 combinations of 9 lipids, 20 best models with the lowest BIC are presented. BIC was calculated from logistic regression with NASH status (NASH vs. NAFL) as an outcome and a combination of 9 lipids identified from the previous 1-lipid model.

Accordingly, in one method of the disclosure, the method comprises obtaining a sample from a subject (e.g., a plasma sample), extracting the bioactive lipids in the sample and determining the levels of at least 14, 15-diHETrE, LPC 0-18:0 and PC 34:4. In another embodiment, the method can include measuring at least 14,15-diHETrE, LPC 0-18:0, PC 34:4 and PE 38:0, PE 0-40:7. In another embodiment, the method can include measuring at least 14, 15-diHETrE, LPC 0-18:0, PC 34:4, and LPC 20:5. In another embodiment, the method can include measuring at least 14, 15-diHETrE, LPC 0-18:0, PC 34:4, and PC 36:5. In another embodiment, the method can include measuring at least 14, 15-diHETrE, LPC 0-18:0, PC 34:4, and PC 40:8, PC 0-40:1. It is to be understood that the disclosure contemplates measuring the 20 possible combinations of Table B. The measurements are compared to a control or reference level (e.g., levels associated with a subject lacking NASH), wherein a statistically significant difference in the markers is indicative of NASH. Moreover, it will be recognized that the reference level will be a reference level for the particular type of measurement used.

The disclosure provides a substantially non-invasive method of predicting or assessing the risk of progression of liver disease in a patient comprising obtaining a plasma sample from a subject and optionally treating the plasma sample with alcohol to dissolve free eicosanoids and free polyunsaturated fatty acid (fPUFA) to obtain free-dissolved eicosanoids and free-dissolved fPUFAs; purifying bioactive lipids including eicosanoids and PUFAs; measuring the level of bioactive lipids selected from the group consisting of at least 14, 15-diHETrE, LPC 0-18:0 and PC 34:4, and optionally one or more additional compounds selected from the group consisting of LPC 20:5, PE 38:0, PE O-40:7, PC 36:5, PC 40:8, PC 0-40:1 and PC 0-34:4; determining the area under receiver operating characteristic curve (AUROC) based upon a ratio of the levels of the bioactive lipids matched with deuterated internal standards of the same metabolite. In another embodiment, the method can include measuring at least 14,15-diHETrE, LPC 0-18:0, PC 34:4 and PE 38:0, PE 0-40:7. In another embodiment, the method can include measuring at least 14, 15-diHETrE, LPC 0-18:0, PC 34:4, and LPC 20:5. In another embodiment, the method can include measuring at least 14, 15-diHETrE, LPC 0-18: 0, PC 34:4, and PC 36:5. In another embodiment, the method can include measuring at least 14, 15-diHETrE, LPC 0-18:0, PC 34:4, and PC 40:8, PC 0-40:1. In another embodiment, the NAFLD is nonalcoholic steatohepatitis (NASH). In another or further embodiment, the AUROC is about at least 0.8, at least about 0.9, or at least about 0.99.

It is to be understood that while the disclosure has been described in conjunction with specific embodiments thereof, that the foregoing description as well as the examples which follow are intended to illustrate and not limit the scope of the disclosure. Other aspects, advantages and modifications within the scope of the disclosure will be apparent to those skilled in the art to which the disclosure.

### EXAMPLES

### Example 1

Reagents. All reagents are HPLC grade and were purchased from Fisher Scientific.

Clinical and Physiological factors. All samples were drawn in fasting state. A full clinical examination was performed and the appropriate form was filled out at that visit by the site-investigator. A diet history was also available for a subset of these subjects based on the “recall” method. The presence of Type 2 diabetes, dyslipidemia and use of statins and other drugs was captured at the visit.

Pre-analytical processing. Plasma samples were collected from patients and healthy volunteers; the detailed description of the patients in the study population including baseline demographic, clinical, biochemical and histologic characteristics is provided is summarized in Table 1. Patients with NAFLD were diagnosed and confirmed by liver biopsy examination; patients with other causes of liver disease were excluded. All patients underwent a standard history and physical exam, biochemical testing, and the magnetic resonance imaging-estimated proton density fat fraction (MRI-PDFF). On the basis of the liver histology, subjects with NAFLD were divided into two groups, those with NAFL and those with NASH. Plasma samples were collected in heparin-tubes and plasma was separated within 30 minutes of blood-draw. The samples were aliquoted into 0.5 ml tubes, immediately frozen and stored at −70° C. on site. All samples were identified by bar-code technology. Within 1 month of collection, samples were shipped in dry ice from the site to the NASH CRN Biosample Repository (at Fisher Bioservices). Samples were stored frozen at −70° C. Samples were withdrawn upon request from the NASH CRN data coordinating center and shipped directly to the lipidomics analysis facility in frozen state. They were thawed immediately before processing.

Methods for bioactive lipid analysis. General categories of lipids examined in lipidomics analysis are described by Quehenberger and Dennis (N. Engl. J. Med., 365: 1812-23, 2011). Methods for phospholipids (including phospholipids and lysophospholipids) and sphingolipids (including ceramides and sphingomyelin) are standard established ultrahigh performance liquid chromatography/mass spectrometric (UPLC/MS) methods (Quehenberger et al., J. Lipid Res., 51 (11): 3299-305, 2010; and Baker et al., J. Lipid Res., 55:2432-42, 2014). The data was collected and analyzed using a QTRAP 6500 LC/MS/MS system (AB SCIEX, Redwood Shores, CA), which is a hybrid quadrupole-linear ion trap mass spectrometer. Source parameters (e.g., temperatures, gas flows, etc.) were optimized using a mixture of phospholipid and sphingolipid standards that were tee-infused with a syringe pump into the flow of an Acquity ultra performance liquid chromatography (UPLC) system (Waters, Milford, MA) delivering the sample. The plasma samples used for phospholipid and sphingolipid analysis were extracted before analysis using lipid category specific extraction protocols (Harkewicz et al., Ann. Rev. of Biochem., 80:301-25, 2011), including modified Bligh and Dyer (J. Biochem. Physiol, 37:911-917, 1959) and Folch lipid extraction and solid phase extraction protocols (Quehenberger et al., J. Lipid Res., 51 (11): 3299-305, 2010).

Samples were loaded in a random manner to avoid machine bias. Samples were routinely spiked with known amounts of non-endogenous synthetic internal standards. These internal standards consist either of odd chain complex lipid standards that were not present in the native sample or of authentic deuterated standards. After lipid extraction, samples were reconstituted in appropriate solvents specific for each of the lipid categories and the extracts were stored at −70° C. prior to MS analysis. The lipids were separated by normal phase UPLC using a binary solvent elution system. The eluted lipids were interfaced and analyzed were analyzed on a hybrid triple quadrupole/linear ion trap mass spectrometer (ABSciex QTRAP 6500) equipped with a robotic UPLC (Waters Acquity). Molecular lipids were analyzed in both positive and negative ion modes using multiplex technologies that include precursor ion scanning (PIS) and neutral loss (NL) based methods (Barbier et al., Gastroenterology, 124 (7): 1926-40, 2003) as well as multiple reaction monitoring (MRM) approaches (Li et al., Prog. In Phys., 34 (4): 314-8, 2003). Lipid category and class specific internal standards were used for quantifying endogenous lipid species. The mass spectrometry data obtained from MS instruments was exported as .wiff or .txt files that represent the basic raw files of lipidomic analysis. These files contained information on masses of identified molecules and their counts (intensities and areas). Masses and counts of detected peaks were converted into a list of corresponding lipid names and concentrations. Calibration lines were generated to determine the dynamic quantification range for each lipid class monitored, e.g., the quantification limits. As the internal standards used behave in the same way as endogenous lipids, they are used for quantifying endogenous lipid species using the isotope-dilution approach. The calibration lines consisted of a minimum of four accepted standard points covering the linear quantification range. Quantification of lipids was carried out by forming ratios between the endogenous lipids and internal standards. The ratios are then compared with the ratios of exogenous quantification standards that were spiked with internal standards, analyzed under identical conditions as the biological samples and used to generate complete standard curves.

Separation and quantification of Eicosanoids. In certain instances eicosanoids can be analyzed as follows. Separation was performed on an Acquity ultra-performance liquid chromatography (UPLC) system (Waters, Milford, MA, USA), equipped with RP18 column (2.1×100 mm; 1.7 μm; Waters). The mobile phase condition and mass spectrometer parameters are described in Wang et al. (J. Chromatogr., 1359:60-69, 2014). Data was collected on an AB/Sciex 6500 QTRAP hybrid, triple quadrupole mass spectrometry using negative electrospray and scheduled multiple reaction monitoring (MRM) mode. In some embodiments, recovery rates were determined by comparing peak areas using a set of 173 purified standards containing all internal standards before and after treatment with KOH. All determinations were performed in triplicate and the average value reported. The precision of the quantitation was determined by the coefficient of variation (CV), calculated from the mean of three replicates and expressed as the relative standard deviation (&RSD).

Quality Control. Quality control was performed based on the ratio of synthetic Internal Standards (IS) to corresponding post-extract spiked External Standards (ES), and MS analysis of extracted matrix and solvents served as quality controls (QC) of the analysis. In addition, extracted reference plasma samples were analyzed for monitoring the instruments' performance. The analysis acceptance standards were based on the linearity of the calibration lines. The linear regression had to exceed 0.95 based on at least four out of six non-zero standards. The analysis was accepted based on the identification of sample specific IS and ES. The Coefficient of Variation (CV) of an area ratio (cps) of internal to external standards (IS/ES) was used to identify potential technical outliers per the analysis platform.

TABLE 1
Baseline demographic and histological characteristics
of the patients in the study population.

| | Mean (±SD) or N (%) | | | | |
|---|---|---|---|---|---|
| | Healthy control | | NAFLD patients | | |
| | (N = 68) | | (N = 304) | | p* |
| Age (years) | 43.3 | (±17.5) | 49.4 | (±11.8) | <0.001 |
| Age | | | | | <0.001 |
| 18-34 | 30 | (44%) | 44 | (14%) | |
| 35-54 | 15 | (22%) | 152 | (50%) | |
| 55-74 | 23 | (34%) | 108 | (36%) | |
| Sex, male | 12 | (18%) | 105 | (35%) | 0.06 |
| Race | | | | | 0.40 |
| Non-Hispanic white | 48 | (71%) | 235 | (77%) | |
| Non-Hispanic black | 5 | (7%) | 11 | (4%) | |
| Hispanic | 9 | (13%) | 32 | (11%) | |
| Other | 6 | (9%) | 26 | (9%) | |
| BMI (kg/m²) | 26.1 | (±5.5) | 34.5 | (±5.8) | <0.001 |
| BMI category | | | | | <0.001 |
| Underweight | 1 | (1%) | 0 | (0%) | |
| Normal | 36 | (53%) | 9 | (3%) | |
| Overweight | 17 | (25%) | 68 | (22%) | |
| Obese | 14 | (21%) | 226 | (75%) | |
| Type 2 diabetes | 2 | (3%) | 105 | (35%) | <0.001 |
| Bilirubin, total (mg/dL) | 0.4 | (±0.2) | 0.7 | (±0.4) | <0.001 |
| Aspartate aminotransferase, AST (U/L) | 21 | (±6) | 54 | (±39) | <0.001 |
| Alanine aminotransferase, ALT (U/L) | 18 | (±10) | 71 | (±46) | <0.001 |
| Alkaline phosphatase, ALP (U/L) | 68 | (±20) | 91 | (±36) | <0.001 |
| Fibrosis stage^{†} | | | | | |
| 0. None | 68 | (100%) | 108 | (36%) | |
| 1a. Mild, zone 3 perisinusoidal | | | 16 | (5%) | |
| 1b. Moderate, zone 3, perisinusoidal | | | 18 | (6%) | |
| 1c. Portal/periportal only | | | 5 | (2%) | |
| 2. Zone 3 and periportal, any combination | | | 64 | (21%) | |
| 3. Bridging | | | 46 | (15%) | |
| 4. Cirrhosis | | | 47 | (15%) | |
| NASH stage^{†} | | | | | |
| Not NAFLD | 68 | (100%) | 0 | (0%) | |
| 0. NAFL (NAFLD, not NASH) | 0 | (0%) | 81 | (27%) | |
| 1a. borderline NASH, zone 3 pattern | 0 | (0%) | 63 | (21%) | |
| 1b. borderline NASH, zone 1 periportal pattern | 0 | (0% | 1 | (<1%) | |
| 2. Definite NASH | 0 | (0%) | 159 | (52%) | |
| Time difference between lab exam and biopsy (day^{)‡} | | | 74 | (±89) | |

*P-value from student t-test for continuous variables and Fisher's exact test for categorical variables.
^{†}Biopsy not done for healthy controls.
^{‡}Given by date of lab exam − date of biopsy.

Differences between the NAFL and NASH groups were assessed with a Student's t-test. Statistically significant differences for NAFL/NASH with p<0.05 were observed.

TABLE 2
Top 16 lipids with the lowest BIC

| Rank | Class of lipid | Lipid | BIC |
|---|---|---|---|
| 1 | Ceramide | CER P-d18:1/18:0 | 254.8 |
| 2 | Sphingomyelin | SM 36:3 | 264.5 |
| 3 | Phospholipid | LPE 18:1 | 269.2 |
| 4 | Phospholipid | LPC O-18:0 | 269.3 |
| 5 | Sphingomyelin | SM 34:3 | 269.9 |
| 6 | Phospholipid | PC 42:10, PC O-42:3 | 270.6 |
| 7 | Phospholipid | LPC 18:2 | 272.2 |
| 8 | Phospholipid | PC 42:9, PC O-42:2 | 273.9 |
| 9 | Ceramide | CER P-d18:0/18:0 | 274.3 |
| 10 | Eicosanoid | dhk PGD2 | 275.5 |
| 11 | Phospholipid | LPC O-16:0 | 283.3 |
| 12 | Ceramide | CER d18:1/24:1 | 284.6 |
| 13 | Eicosanoid | 5,6-diHETrE | 287.9 |
| 14 | Ceramide | CER P-d18:1/20:5 | 288.4 |
| 15 | Phospholipid | PC 40:0 | 289.5 |
| 16 | Eicosanoid | 5-HETE | 290.4 |

The metabolites derived from AA show slightly increased levels in NAFL and NASH but these increases did not reach significance in this study. Similarly, 9, 10-EpOME, 9, 10-DIHOME, 13-HODE, and 9-oxoODE, all metabolites derived from linoleic acid (LA), showed stepwise increases in NAFL and NASH, compared with controls. Clinically, it is important to be able to distinguish NAFL from NASH and several of these metabolites including 13-HODE and 9-oxoODE were present at higher levels in the plasma from NAFL compared with NASH. In addition, several metabolites derived from DGLA including 8-HETrE and 15-HETrE were also significantly increased in NASH, whereas no differences were found between control and NAFL. Interestingly, the plasma levels of both the omega-3 fatty acid DHA and its anti-inflammatory metabolite 17-HDoHE were significantly increased in NASH.

During the preliminary UPLC/MS/MS method development, different concentrations of KOH (0.20-1.31M) and BHT (0 to 10 mM) in the sample extract solution were compared. Based on the quality of peak shape and resolution of metabolites, it was confirmed that about 0.20 to 0.66 M KOH (e.g., about 0.10-0.70 M KOH) and about 2.5 mM BHT (e.g., about 2.0 to 3.0 mM BHT) in the lipid extraction solution yielded the optimal results. Higher base results in too much eicosanoid degradation, and lesser gives insufficient hydrolysis of esterified oxidized complex lipids. Too high a BHT concentration produces crystallization. In another embodiment, the amount of BHT is 2.5 mM. During the process and prior to the SPE column, the extract was diluted with H₂O to avoid too high a salt concentration during the SPE extract.

The findings from the NAFLD samples relate to the identification of specific fatty acid oxidation products as potential novel, systemic, noninvasive markers to differentiate NASH from NAFL. The concentrations of LA, AA, DGLA, EPA and DHA derivatives from enzymatic and free radical pathways in the plasma of patients with NAFLD and healthy individuals were evaluated.

Many of the PUFA products are much more elevated in NAFL and NASH subjects compared to control. Lipid peroxidation products such as HODEs originating from the conversion of LA and HETEs originating from the conversion of AA in reactions catalyzed by cellular lipoxygenases were increased in the liver during peroxidation in association with the increase in triglyceride. The plasma concentrations of proinflammatory eicosanoids including 5-HETE, 8-HETE, 11-HETE, 15-HETE, 13-HODE, and 9-oxoODE are much more elevated in NAFL patients compared to NASH patients and control subjects. The decrease in NASH indicated some of the eicosanoids were degraded into others.

Interestingly, the omega-3 fatty acid DHA (p<0.001) and its metabolite 17-HDoHE (p<0.0001), were significantly increased in NASH compared with NAFL and control. The latter metabolite is of particular interest as it is a precursor for protectins, a group of lipid mediators with anti-inflammatory properties.

### Example 2

Study samples. Blood plasma samples from adult and pediatric patients with varying phenotypes of NAFLD were obtained from the NIDDK Nonalcoholic Steatohepatitis Clinical Research Network (NASH CRN) NAFLD Database prospective cohort study (NAFLD group); and samples of healthy control subjects were obtained from cohort studies at the University of California, San Diego (UCSD). Plasma samples were utilized for lipid measurement using multiplex technologies based on UPLC-mass spectrometry as described above. A total of 131 lipids were detected, which include 65 eicosanoids, 16 sterols, 37 ceramides, and 13 sphingomyelins. NAFLD patients were older than health controls (49.4 vs. 43.3 years; P<0.001); the proportion of male was higher (35% vs. 18%; P=0.06); and BMI was higher (26.1 vs. 34.5 kg/m²; P<0.001) (Table 1). In NAFLD patients, as compared to healthy controls, total bilirubin, AST, ALT, and ALP were higher (Table 1).

Statistical analysis. Among the 280 lipids that were detected, 62 lipids were dropped that had 210% non-detectable values among total samples from the analyses. For the remaining 218 lipids (23 eicosanoids, 15 sterols, 37 ceramides, 13 sphingomyelins, and 130 phospholipids), non-detectable values were imputed, if any, with the 1/5 of the lower limit of detection. As a method to manage extreme values, each lipid was winsorized by replacing values of 3 most extreme positions with the value of the 4th extreme position in both high and low ends.

Demographics, physical, and clinical indicators of study participants were compared by NAFLD status (NAFLD case vs. healthy control) using the t-test for continuous variables and Fisher's exact test for categorical variables. Distributions of lipids were assessed by NAFLD status using histograms.

Bayesian Information Criterion (BIC) was used to select lipids for constructing diagnostic models. BIC was derived from logistic regression models of NAFLD status in relation to each lipid. Second, among the 16 lipids with the lowest BIC, the final model was selected among all their combinations using BIC. The best 16 of the 218 lipids ranked by the statistical information provided for discriminating NAFLD cases from healthy controls using the Bayesian Information Criteria (BICs) were selected, with lower BICs indicating higher information provided. We calculated BIC using logistic regression with the NAFLD status as an outcome and each lipid as a covariate. The best multi-lipid regression model for NAFLD case vs. healthy control as the combination of the 16 selected lipids that maximized model information (lowest BIC) from the set of all possible 216=65,536 logistic regression models were chosen. Since, p-values are not necessary to choose the best model, no multiplicity adjustments are needed.

Leave-one-out cross-validation was used to examine the performance of the selected model. Used indicators were: area under receiver operating characteristic curve (AUROC); sensitivity and specificity at fixed sensitivity and specificity and at the maximum Youden's index; and positive and negative predicted values (PPV and NPV) at varying prevalence.

Among 218 lipids, whose proportion of non-detectable values was less than 10%, 137 lipids (63%) had BIC less than 359.8 of the null model (Table 3). Top 16 lipids, which we used for the next best subsets analysis, consisted of 3 eicosanoids, 4 ceramides, 2 sphingomyelins, and 7 phospholipids (Table 4). Among their 216=35, 536 combinations, the best model consisted of dhk PDG2, 5-HETE, ceramide Pd18: 1/20:5, sphingomyelin 34:3, phosphatidylcholine 42: 9, 0-42:2, lyso-phosphatidylethanolamine 18:1 (Table 5). In the top 20 best models, dhk PDG2, 5-HETE, ceramide Pd18: 1/20:5, and either sphingomyelin 36:3 or 34:3 appeared constantly (Table A).

TABLE 3
BIC and area under the ROC curve (AUROC) from the
lipid model*, lipids sorted by BIC (218 lipids)

| Rank | Class of lipid | Lipid | BIC | AUROC |
|---|---|---|---|---|
| 1 | Ceramide | CER P-d18:1/18:0 | 254.8 | 0.874 |
| 2 | Sphingomyelin | SM 36:3 | 264.5 | 0.863 |
| 3 | Phospholipid | LPE 18:1 | 269.2 | 0.849 |
| 4 | Phospholipid | LPC O-18:0 | 269.3 | 0.858 |
| 5 | Sphingomyelin | SM 34:3 | 269.9 | 0.851 |
| 6 | Phospholipid | PC 42:10, PC O-42:3 | 270.6 | 0.871 |
| 7 | Phospholipid | LPC 18:2 | 272.2 | 0.891 |
| 8 | Phospholipid | PC 42:9, PC O-42:2 | 273.9 | 0.845 |
| 9 | Ceramide | CER P-d18:0/18:0 | 274.3 | 0.846 |
| 10 | Eicosanoid | dhk PGD2 | 275.5 | 0.844 |
| 11 | Phospholipid | LPC O-16:0 | 283.3 | 0.866 |
| 12 | Ceramide | CER d18:1/24:1 | 284.6 | 0.820 |
| 13 | Eicosanoid | 5,6-diHETrE | 287.9 | 0.773 |
| 14 | Ceramide | CER P-d18:1/20:5 | 288.4 | 0.820 |
| 15 | Phospholipid | PC 40:0 | 289.5 | 0.815 |
| 16 | Eicosanoid | 5-HETE | 290.4 | 0.740 |
| 17 | Phospholipid | LPC 18:1 | 291.4 | 0.840 |
| 18 | Ceramide | CER P-d18:1/16:0 | 296.8 | 0.813 |
| 19 | Phospholipid | PC 42:8, PC O-42:1 | 297.0 | 0.797 |
| 20 | Phospholipid | LPC 16:0 | 299.5 | 0.795 |
| 21 | Ceramide | CER P-d18:0/16:1 | 300.8 | 0.797 |
| 22 | Ceramide | CER P-d18:0/16:0 | 301.0 | 0.818 |
| 23 | Ceramide | CER P-d18:1/18:1 | 301.1 | 0.789 |
| 24 | Ceramide | CER d18:1/20:1 | 302.0 | 0.785 |
| 25 | Sterol | 7,27-dihydroxy-cholesterol | 302.7 | 0.780 |
| 26 | Phospholipid | PC 42:0 | 305.0 | 0.790 |
| 27 | Phospholipid | PC 42:11, PC O-42:4 | 305.3 | 0.812 |
| 28 | Ceramide | CER d18:0/24:1 | 305.9 | 0.776 |
| 29 | Phospholipid | LPE 18:2 | 308.7 | 0.781 |
| 30 | Phospholipid | PC 32:1 | 311.4 | 0.786 |
| 31 | Ceramide | CER d18:1/22:5 | 312.9 | 0.768 |
| 32 | Ceramide | CER d18:1/22:6 | 313.2 | 0.755 |
| 33 | Phospholipid | LPE 18:0 | 313.2 | 0.726 |
| 34 | Sterol | 25-hydroxy-cholesterol | 313.3 | 0.764 |
| 35 | Sterol | Lanosterol | 315.8 | 0.764 |
| 36 | Ceramide | CER d18:1/22:1 | 316.4 | 0.772 |
| 37 | Ceramide | CER d18:1/22:0 | 316.5 | 0.748 |
| 38 | Phospholipid | PC 42:7, PC O-42:0 | 316.7 | 0.758 |
| 39 | Phospholipid | PC 40:1 | 318.1 | 0.770 |
| 40 | Phospholipid | PC O-42:9 | 318.2 | 0.769 |
| 41 | Sphingomyelin | SM 36:1 | 318.2 | 0.701 |
| 42 | Phospholipid | LPC 18:0 | 318.6 | 0.762 |
| 43 | Ceramide | CER d18:1/18:1 | 319.4 | 0.721 |
| 44 | Ceramide | CER d18:1/22:4 | 321.4 | 0.746 |
| 45 | Ceramide | CER P-d18:0/18:1 | 322.1 | 0.743 |
| 46 | Ceramide | CER d18:0/22:0 | 322.5 | 0.739 |
| 47 | Phospholipid | LPC 20:4 | 325.7 | 0.749 |
| 48 | Ceramide | CER P-d18:1/16:1 | 326.7 | 0.727 |
| 49 | Phospholipid | PC 40:2 | 328.2 | 0.742 |
| 50 | Ceramide | CER d18:1/20:3 | 328.2 | 0.718 |
| 51 | Ceramide | CER d18:1/20:4 | 328.6 | 0.725 |
| 52 | Phospholipid | PC 42:4 | 328.7 | 0.744 |
| 53 | Ceramide | CER d18:1/26:1 | 329.7 | 0.714 |
| 54 | Phospholipid | PE 30:1 | 329.8 | 0.725 |
| 55 | Phospholipid | LPC 22:0 | 329.9 | 0.755 |
| 56 | Phospholipid | LPE 20:4 | 330.5 | 0.716 |
| 57 | Eicosanoid | 9-HOTrE | 330.9 | 0.718 |
| 58 | Phospholipid | PC O-40:5 | 331.1 | 0.707 |
| 59 | Phospholipid | PC O-42:10 | 332.8 | 0.707 |
| 60 | Phospholipid | PC O-40:6 | 334.0 | 0.694 |
| 61 | Phospholipid | PC O-40:4 | 334.1 | 0.718 |
| 62 | Phospholipid | PC O-38:5 | 334.5 | 0.685 |
| 63 | Ceramide | CER d18:0/24:0 | 336.8 | 0.687 |
| 64 | Phospholipid | LPC 22:5 | 337.1 | 0.705 |
| 65 | Ceramide | CER d18:1/18:0 | 337.6 | 0.653 |
| 66 | Ceramide | CER d18:1/24:0 | 339.0 | 0.671 |
| 67 | Ceramide | CER d18:1/20:0 | 339.0 | 0.660 |
| 68 | Phospholipid | LPE 16:0 | 339.4 | 0.673 |
| 69 | Phospholipid | PE 40:6 | 339.4 | 0.703 |
| 70 | Phospholipid | PC 42:5 | 339.6 | 0.718 |
| 71 | Phospholipid | LPC 22:4 | 340.3 | 0.686 |
| 72 | Phospholipid | LPC 22:6 | 341.0 | 0.708 |
| 73 | Phospholipid | PE 42:9 | 341.6 | 0.730 |
| 74 | Phospholipid | LPC 22:3 | 342.2 | 0.675 |
| 75 | Sterol | Cholestanol | 342.7 | 0.626 |
| 76 | Phospholipid | PC O-38:3 | 342.7 | 0.647 |
| 77 | Phospholipid | PE 32:1 | 342.7 | 0.680 |
| 78 | Phospholipid | LPC 16:1 | 343.0 | 0.714 |
| 79 | Phospholipid | PE 38:6 | 343.7 | 0.675 |
| 80 | Phospholipid | LPE 22:6 | 343.9 | 0.690 |
| 81 | Phospholipid | PC 40:8, PC O-40:1 | 344.4 | 0.667 |
| 82 | Phospholipid | PC O-36:2 | 344.4 | 0.680 |
| 83 | Sphingomyelin | SM 38:2 | 345.8 | 0.659 |
| 84 | Phospholipid | PC O-38:6 | 346.0 | 0.633 |
| 85 | Phospholipid | PE 40:7, PE O-40:0 | 347.3 | 0.688 |
| 86 | Phospholipid | PC O-34:0 | 347.8 | 0.654 |
| 87 | Eicosanoid | 9-oxoODE | 348.8 | 0.668 |
| 88 | Eicosanoid | 9,10 diHOME | 349.1 | 0.582 |
| 89 | Phospholipid | LPC 20:5 | 349.4 | 0.702 |
| 90 | Phospholipid | PC O-42:11 | 349.6 | 0.640 |
| 91 | Ceramide | CER P-d18:1/20:4 | 349.7 | 0.616 |
| 92 | Eicosanoid | 13-HODE | 349.9 | 0.633 |
| 93 | Phospholipid | PE 40:5 | 349.9 | 0.688 |
| 94 | Ceramide | CER P-d18:0/20:5 | 350.1 | 0.636 |
| 95 | Phospholipid | LPC 20:3 | 350.2 | 0.757 |
| 96 | Phospholipid | PC O-36:3 | 350.3 | 0.648 |
| 97 | Phospholipid | PI 38:5 | 350.3 | 0.664 |
| 98 | Sphingomyelin | SM 36:4 | 350.4 | 0.648 |
| 99 | Phospholipid | PE 38:5 | 350.7 | 0.658 |
| 100 | Sterol | Dihydro-lanosterol | 350.9 | 0.598 |
| 101 | Phospholipid | PC O-36:4 | 350.9 | 0.589 |
| 102 | Phospholipid | PC 40:3 | 350.9 | 0.637 |
| 103 | Eicosanoid | 12,13 diHOME | 351.0 | 0.585 |
| 104 | Ceramide | CER d18:0/16:0 | 351.2 | 0.557 |
| 105 | Phospholipid | PC O-34:1 | 351.4 | 0.643 |
| 106 | Phospholipid | PC O-34:2 | 351.7 | 0.632 |
| 107 | Phospholipid | PS 38:4 | 351.7 | 0.387 |
| 108 | Ceramide | CER P-d18:1/20:3 | 351.9 | 0.609 |
| 109 | Ceramide | CER P-d18:1/24:1 | 351.9 | 0.615 |
| 110 | Phospholipid | PE 40:9, PE O-40:2 | 352.0 | 0.685 |
| 111 | Phospholipid | PS 40:5 | 352.5 | 0.519 |
| 112 | Ceramide | CER d18:1/16:0 | 353.7 | 0.548 |
| 113 | Phospholipid | PE 34:1 | 353.7 | 0.642 |
| 114 | Phospholipid | PC 38:2, PC O-40:9 | 353.8 | 0.638 |
| 115 | Phospholipid | PS 38:3 | 353.9 | 0.426 |
| 116 | Phospholipid | PC 32:0 | 354.2 | 0.634 |
| 117 | Phospholipid | PE 36:0, PE O-38:7 | 354.4 | 0.578 |
| 118 | Phospholipid | PC 32:2 | 354.6 | 0.594 |
| 119 | Sterol | Campesterol | 355.4 | 0.656 |
| 120 | Phospholipid | PE 34:3 | 355.5 | 0.631 |
| 121 | Phospholipid | PE 38:7 | 356.0 | 0.676 |
| 122 | Phospholipid | PC 42:6 | 356.3 | 0.625 |
| 123 | Phospholipid | PC 40:5 | 356.3 | 0.625 |
| 124 | Phospholipid | PC 40:6 | 356.4 | 0.614 |
| 125 | Sterol | 24-hydroxy-cholesterol | 356.6 | 0.599 |
| 126 | Phospholipid | PC O-36:0 | 356.6 | 0.639 |
| 127 | Phospholipid | PC O-36:5 | 356.8 | 0.475 |
| 128 | Eicosanoid | 9-HODE | 357.0 | 0.545 |
| 129 | Phospholipid | PC O-36:1 | 357.1 | 0.635 |
| 130 | Ceramide | CER d18:1/26:0 | 357.1 | 0.588 |
| 131 | Phospholipid | PS 36:1 | 357.5 | 0.388 |
| 132 | Phospholipid | PE 40:8, PE O-40:1 | 357.5 | 0.677 |
| 133 | Phospholipid | PE 34:2 | 357.5 | 0.631 |
| 134 | Phospholipid | PE 42:10 | 357.9 | 0.650 |
| 135 | Ceramide | CER P-d18:1/22:0 | 358.0 | 0.664 |
| 136 | Phospholipid | PC 38:5 | 358.4 | 0.596 |
| 137 | Phospholipid | PC 36:3 | 359.7 | 0.595 |
| 138 | Phospholipid | PE 34:0 | 359.8 | 0.620 |
| 139 | Eicosanoid | 15-HETTE | 360.2 | 0.634 |
| 140 | Eicosanoid | 20cooh AA | 360.2 | 0.563 |
| 141 | Phospholipid | PE 38:3 | 360.7 | 0.637 |
| 142 | Eicosanoid | 19,20 DiHDPA | 361.6 | 0.603 |
| 143 | Phospholipid | PC 34:3 | 361.7 | 0.582 |
| 144 | Phospholipid | PE 42:8 | 361.7 | 0.652 |
| 145 | Phospholipid | PC O-40:3 | 361.8 | 0.536 |
| 146 | Phospholipid | PC O-42:5 | 361.9 | 0.580 |
| 147 | Sphingomyelin | SM 34:2 | 362.0 | 0.562 |
| 148 | Sterol | 7a-hydroxy-4-cholesten-3-one | 362.0 | 0.596 |
| 149 | Phospholipid | PC 40:4 | 362.1 | 0.577 |
| 150 | Phospholipid | PE 36:4 | 362.5 | 0.597 |
| 151 | Phospholipid | PC 34:4 | 362.5 | 0.583 |
| 152 | Sterol | Desmosterol | 362.6 | 0.562 |
| 153 | Phospholipid | PC 40:7, PC O-40:0 | 362.7 | 0.552 |
| 154 | Phospholipid | PI 36:1 | 362.7 | 0.579 |
| 155 | Sphingomyelin | SM 32:2 | 362.8 | 0.562 |
| 156 | Phospholipid | PE 38:4 | 362.9 | 0.609 |
| 157 | Eicosanoid | 11,12-diHETrE | 363.1 | 0.583 |
| 158 | Phospholipid | PE 36:5 | 363.2 | 0.600 |
| 159 | Phospholipid | PS 36:2 | 363.2 | 0.345 |
| 160 | Phospholipid | PI 34:2 | 363.2 | 0.581 |
| 161 | Phospholipid | PE 40:4 | 363.3 | 0.649 |
| 162 | Phospholipid | PI 38:4 | 363.4 | 0.525 |
| 163 | Eicosanoid | 12-HETE | 363.4 | 0.407 |
| 164 | Eicosanoid | 14,15-diHETrE | 363.4 | 0.546 |
| 165 | Phospholipid | PE 42:7 | 363.4 | 0.603 |
| 166 | Eicosanoid | tetranor 12-HETE | 363.5 | 0.566 |
| 167 | Phospholipid | PC 38:4 | 363.9 | 0.539 |
| 168 | Phospholipid | PC O-38:2 | 364.0 | 0.400 |
| 169 | Sterol | 7-dehydrocholesterol | 364.1 | 0.628 |
| 170 | Phospholipid | PE 38:0, PE O-40:7 | 364.2 | 0.494 |
| 171 | Phospholipid | PI 36:3 | 364.2 | 0.546 |
| 172 | Phospholipid | PC 38:6 | 364.2 | 0.521 |
| 173 | Phospholipid | PC 38:0, PC O-40:7 | 364.2 | 0.496 |
| 174 | Phospholipid | PE 36:3 | 364.2 | 0.591 |
| 175 | Eicosanoid | 12,13 EpOME | 364.3 | 0.438 |
| 176 | Phospholipid | PC 34:0, PC O-36:7 | 364.3 | 0.546 |
| 177 | Sphingomyelin | SM 34:0 | 364.3 | 0.519 |
| 178 | Phospholipid | PE 38:1, PE O-40:8 | 364.4 | 0.421 |
| 179 | Ceramide | CER P-d18:1/24:0 | 364.5 | 0.498 |
| 180 | Sphingomyelin | SM 34:1 | 364.5 | 0.492 |
| 181 | Eicosanoid | 14 HDoHE | 364.5 | 0.449 |
| 182 | Sphingomyelin | SM 36:2 | 364.6 | 0.477 |
| 183 | Sphingomyelin | SM 32:0 | 364.6 | 0.542 |
| 184 | Phospholipid | PC O-34:3 | 364.7 | 0.419 |
| 185 | Phospholipid | PE 36:2 | 364.8 | 0.589 |
| 186 | Eicosanoid | 15-HETE | 364.8 | 0.459 |
| 187 | Phospholipid | PC 36:4 | 364.9 | 0.530 |
| 188 | Phospholipid | PC 36:0, PC O-38:7 | 364.9 | 0.515 |
| 189 | Sphingomyelin | SM 32:1 | 365.0 | 0.536 |
| 190 | Phospholipid | PE 38:2, PE O-40:9 | 365.0 | 0.608 |
| 191 | Phospholipid | PI 34:1 | 365.0 | 0.529 |
| 192 | Phospholipid | PC 36:1 | 365.0 | 0.508 |
| 193 | Eicosanoid | 11-HETE | 365.1 | 0.428 |
| 194 | Phospholipid | PI 38:3 | 365.1 | 0.545 |
| 195 | Phospholipid | PI 36:4 | 365.2 | 0.502 |
| 196 | Phospholipid | PE 36:1 | 365.3 | 0.605 |
| 197 | Eicosanoid | 16 HDoHE | 365.3 | 0.475 |
| 198 | Phospholipid | PE 32:2 | 365.4 | 0.536 |
| 199 | Eicosanoid | 8-HETE | 365.4 | 0.453 |
| 200 | Phospholipid | PC 38:3 | 365.4 | 0.525 |
| 201 | Phospholipid | PC 38:1, PC O-40:8 | 365.4 | 0.534 |
| 202 | Sterol | 4β-hydroxycholesterol | 365.5 | 0.528 |
| 203 | Sterol | 7a-hydroxy-cholesterol | 365.5 | 0.519 |
| 204 | Phospholipid | PC O-34:4 | 365.5 | 0.518 |
| 205 | Phospholipid | PC 34:2 | 365.5 | 0.533 |
| 206 | Phospholipid | PC 36:5 | 365.6 | 0.498 |
| 207 | Phospholipid | PI 36:2 | 365.6 | 0.510 |
| 208 | Phospholipid | PC 36:2 | 365.6 | 0.492 |
| 209 | Eicosanoid | 9,10 EpOME | 365.6 | 0.606 |
| 210 | Ceramide | CER P-d18:0/22:1 | 365.6 | 0.575 |
| 211 | Sterol | 27-hydroxy-cholesterol | 365.6 | 0.515 |
| 212 | Sterol | Sitosterol | 365.6 | 0.428 |
| 213 | Phospholipid | PC 34:1 | 365.7 | 0.515 |
| 214 | Sphingomyelin | SM 34:4 | 365.7 | 0.524 |
| 215 | Sterol | 14-demethyl-lanosterol | 365.7 | 0.490 |
| 216 | Ceramide | CER P-d18:1/22:1 | 365.7 | 0.542 |
| 217 | Phospholipid | PE 32:0 | 365.7 | 0.526 |
| 218 | Phospholipid | PC O-36:6 | 365.7 | 0.499 |

*The binary outcome (NAFLD case vs. healthy control) was regressed on each lipid using simple logistic regression.

TABLE 4
Top 16 lipids with the lowest BIC from the 1-lipid model*†

| Rank | Class of lipid | Lipid | BIC |
|---|---|---|---|
| 1 | Ceramide | CER P-d18:1/18:0 | 254.8 |
| 2 | Sphingomyelin | SM 36:3 | 264.5 |
| 3 | Phospholipid | LPE 18:1 | 269.2 |
| 4 | Phospholipid | LPC O-18:0 | 269.3 |
| 5 | Sphingomyelin | SM 34:3 | 269.9 |
| 6 | Phospholipid | PC 42:10, PC O-42:3 | 270.6 |
| 7 | Phospholipid | LPC 18:2 | 272.2 |
| 8 | Phospholipid | PC 42:9, PC O-42:2 | 273.9 |
| 9 | Ceramide | CER P-d18:0/18:0 | 274.3 |
| 10 | Eicosanoid | dhk PGD2 | 275.5 |
| 11 | Phospholipid | LPC O-16:0 | 283.3 |
| 12 | Ceramide | CER d18:1/24:1 | 284.6 |
| 13 | Eicosanoid | 5,6-diHETrE | 287.9 |
| 14 | Ceramide | CER P-d18:1/20:5 | 288.4 |
| 15 | Phospholipid | PC 40:0 | 289.5 |
| 16 | Eicosanoid | 5-HETE | 290.4 |

*BIC was calculated from logistic regression with the NAFLD status (NAFLD case vs. healthy control) as an outcome and each lipid as a covariate. The lower BIC is, the higher the model provides information.
†Results of all the assessed lipids are shown in Table 5.

TABLE 5
Selected final model*^{†‡} (N = 372)

| | OR | | Standardized OR^{§} | | p |
|---|---|---|---|---|---|
| dhk PDG2 (pmol/mL) | 1.96 | (1.45, 2.66) | 72.6 | (10.5, 501) | <0.001 |
| Ceramide Pd18:1/20:5 (NI/mL) | 5.55 | (2.16, 14.2) | 34.9 | (4.94, 246) | <0.001 |
| Sphingomyelin 34:3 (NI/mL) | 157 | (8.35, 2970) | 11.8 | (2.82, 49.4) | 0.001 |
| Lyso-phosphatidylethanolamine 18:1 (NI/mL) | 0.74 | (0.61, 0.91) | 0.17 | (0.05, 0.57) | 0.004 |
| Phosphatidylcholine 42:9, O-42:2 (NI/mL) | 0.56 | (0.41, 0.76) | 0.14 | (0.05, 0.39) | <0.001 |
| 5-HETE (pmol/mL) | 0.46 | (0.32, 0.66) | 0.11 | (0.04, 0.30) | <0.001 |

NI/mL = normalized intensity relative to an internal standard/mL; OR = odds ratio.
*Among the best 16 lipids with the lowest BIC in the 1-lipid model, all the combinations of these lipids, a total of 2¹⁶ = 65,536 combinations, were compared for BIC using multiple regression models in which multiple lipids were included as covariates. The model that yielded the lowest BIC was selected as the final model.
^{†}Twenty best models are shown in Table A.
^{‡}The equation is:

[symbol]

[symbol]

[symbol]

[symbol]

^{§}OR associated with an increase of lipid by 1 SD. [symbol]
indicates data missing or illegible when filed

The best model had an AUROC of 0.989 (95% confidence interval (CI)=0.973, 0.997) and a sensitivity of 96% (93, 98) at 95% specificity (Table 6). The positive and negative predictive values (PPV and NPV) were 71% and 99% at 10% NAFLD prevalence, and 90% and 98% at 30% NAFLD prevalence.

TABLE 6
Diagnostic performance of the final model. Leave-one-out cross-
validated AUROC and other performance indicators (N = 372)

| | AUROC | Sensitivity (%) | Specificity (%) | Cutoff |
|---|---|---|---|---|
| | (95% CI) | (95% CI) | (95% CI) | probability |
| At 95% specificity^{†} | | 95.7 | — | 0.892 |
| | | (92.8, 97.7) | | |
| At 95% sensitivity^{†} | 0.989 | — | 97.1 | 0.906 |
| | (0.973, 0.997) | | (89.8, 99.6) | |
| At maximum Youden's index | | 94.1 | 98.5 | 0.939 |
| | | (90.8, 96.5) | (92.1, 1.00) | |

^{†}Sensitivity and specificity fixed at ≥95% and closest to 95.

One model consisted of 13, 14-dihydro-15-keto prostaglandin D2 (dhk-PGD2), 5-HETE, ceramide Pd18:1/20:5, sphingomyelin 34:3, phosphatidylcholine 42:9/0-42:2, lyso-phosphatidylethanolamine 18:1 with an area under the receiver operating characteristics curve (AUROC) of 0.989 (95% confidence interval (CI)=0.973, 0.997). The sensitivity was 96% (95% CI=93, 98) at 95% specificity, and the positive and the negative predicted values (PPV, NPV) were 90% and 98% at NAFLD prevalence of 30%. This model used six lipids and showed high discriminatory performance between NAFLD and healthy controls.

### Example 3

To investigate lipidomic data to identify a minimal set of analytes that discriminate NASH (nonalcoholic hepatosteatosis) from NAFL (nonalcoholic fatty liver blood plasma samples from adult patients with varying phenotypes of NAFLD were obtained from the NIDDK Nonalcoholic Steatohepatitis Clinical Research Network (NASH CRN) NAFLD Database prospective cohort study (N=304). Plasma samples were utilized for lipid measurement of eicosanoids, sterols, ceramides, sphingomyelins, and phospholipids using multiplex technologies based on UPLC-mass spectrometry. A Bayesian Information Criterion (BIC) analysis was used to select lipids for constructing diagnostic models.

Blood plasma samples were obtained from adult patients with varying phenotypes of NAFLD from the NIDDK Nonalcoholic Steatohepatitis Clinical Research Network (NASH CRN) NAFLD Database prospective cohort study. A total of 304 samples consisted of 81 NAFL and 223 NASH cases (64 borderline NASH and 159 definite NASH).

### Statistical Analysis

Among 280 lipids that were detected, 62 lipids were dropped that had 210% non-detectable values among total samples from the analyses. For the remaining 218 lipids (23 eicosanoids, 15 sterols, 37 ceramides, 13 sphingomyelins, and 130 phospholipids), non-detectable values were imputed, if any, with the 1/5 of the lower limit of detection. As a method to manage extreme values, each lipid was winsorized by replacing values of 3 most extreme positions with the value of the 4th extreme position in both high and low ends.

Demographics, physical, and clinical indicators of study participants were compared by NASH status (NASH vs. NAFL) using the t-test for continuous variables and Fisher's exact test for categorical variables. Lipids distributions were presented using histograms.

The best 9 of the 218 lipids ranked by the statistical information provided for discriminating NASH cases from NAFL cases using the Bayesian Information Criteria (BICs) were selected, with lower BICs indicating higher information provided. BIC was calculated using logistic regression with the NASH status as an outcome and each lipid as a covariate. The best multi-lipid regression model for NASH case vs. NAFL was identified as the combination of the 9 selected lipids that maximized model information (lowest BIC) from the set of all possible 29=512 logistic regression models.

Leave-one-out cross-validation was used to examine the performance of the selected model. Used indicators were: area under receiver operating characteristic curve (AUROC); sensitivity and specificity at fixed sensitivity and specificity and at the maximum Youden's index; and positive and negative predicted values (PPV and NPV) at varying prevalence.

### Subject Analysis

NASH and NAFL patients were similar in age (mean: 50.0 vs. 47.8 years), sex (male proportion: 34% vs. 378), and BMI distribution (mean: 34.8 vs. 33.6 kg/m²) (Table 7). In NASH patients, as compared to NAFL patients: type 2 diabetic was more common (42% vs. 268, P<0.001); AST and ALT were elevated (AST: 59 vs. 40 U/L, P<0.001; ALT: 75 vs. 60 U/L, P=0.02); and fibrosis stage was higher (P<0.001; proportion of any fibrosis: 69% vs. 58; proportion of advanced fibrosis: 42% vs. 08).

TABLE 7
Patient characteristics by NASH status (N = 304)

| | Mean (±SD) or N (%) | | | | |
|---|---|---|---|---|---|
| | NAFL patients | | NASH patients | | |
| | (N = 81) | | (N = 223) | | p* |
| Age (years) | 47.8 | (±12.4) | 50.0 | (±11.5) | 0.16 |
| Age | | | | | 0.60 |
| 18-34 | 14 | (17%) | 30 | (13%) | |
| 35-54 | 41 | (51%) | 111 | (50%) | |
| 55-74 | 26 | (32%) | 82 | (37%) | |
| Sex, male | 30 | (37%) | 75 | (34%) | 0.59 |
| Race | | | | | 0.88 |
| Non-Hispanic white | 62 | (77%) | 173 | (78%) | |
| Non-Hispanic black | 2 | (2%) | 9 | (4%) | |
| Hispanic | 10 | (12%) | 22 | (10%) | |
| Other | 7 | (9%) | 19 | (9%) | |
| BMI (kg/m²) | 33.6 | (±5.8) | 34.8 | (±5.8) | 0.14 |
| BMI category | | | | | 0.22 |
| Normal | 2 | (2%) | 7 | (3%) | |
| Overweight | 24 | (30%) | 44 | (20%) | |
| Obese | 55 | (68%) | 171 | (77%) | |
| Type 2 diabetes | 12 | (15%) | 93 | (42%) | <0.001 |
| Bilirubin, total (mg/dL) | 0.7 | (±0.4) | 0.7 | (±0.4) | 0.99 |
| Aspartate aminotransferase, AST (U/L) | 40 | (±26) | 59 | (±41) | <0.001 |
| Alanine aminotransferase, ALT (U/L) | 60 | (±41) | 75 | (±47) | 0.02 |
| Alkaline phosphatase, ALP (U/L) | 87 | (±39) | 93 | (±35) | 0.25 |
| Fibrosis stage^{†} | | | | | <0.001 |
| 0. None | 77 | (95%) | 31 | (14%) | |
| 1a. Mild, zone 3 perisinusoidal | 1 | (1%) | 15 | (7%) | |
| 1b. Moderate, zone 3, perisinusoidal | 0 | (0%) | 18 | (8%) | |
| 1c. Portal/periportal only | 3 | (4%) | 2 | (1%) | |
| 2. Zone 3 and periportal, any | 0 | (0%) | 64 | (29%) | |
| combination | | | | | |
| 3. Bridging | 0 | (0%) | 46 | (21%) | |
| 4. Cirrhosis | 0 | (0%) | 47 | (21%) | |
| NASH stage^{†} | | | | | — |
| 0. NAFL (NAFLD, not NASH) | 81 | (100%) | 0 | (0%) | |
| 1a. borderline NASH, zone 3 pattern | 0 | (0%) | 63 | (28%) | |
| 1b. borderline NASH, zone 1 periportal | 0 | (0%) | 1 | (<1%) | |
| pattern | | | | | |
| 2. Definite NASH | 0 | (0%) | 159 | (71%) | |
| Time difference between lab exam and | 79 | (±116) | 72 | (±76) | 0.56 |
| biopsy (day)^{‡} | | | | | |

*P-value from student t-test for continuous variables and Fisher's exact test for categorical variables.
^{†}Biopsy not done for healthy controls.
^{‡}Given by date of lab exam − date of biopsy.

### Lipid Analysis

Among 218 lipids, whose proportion of non-detectable values was less than 108, 9 lipids (48) had BIC less than 354.5 of the null model (Table 8). Top 9 lipids, which we used for the next best subsets analysis, consisted of 2 eicosanoids and 7 phospholipids (Table 9). Among their 29=512 combinations, the best model consisted of 14, 15-diHETrE, lyso-phosphatidylcholine 0-18:0, and phosphatidylcholine 34:4 (Table 10). In the top 20 best models, 14, 15-diHETrE and lyso-phosphatidylcholine 0-18:0 appeared more constantly than other 7 lipids (Table B).

TABLE 8
BIC and area under the ROC curve (AUROC) from the
1-lipid model*, lipids sorted by BIC (218 lipids)

| Rank | Class of lipid | Lipid | BIC | AUROC |
|---|---|---|---|---|
| 1 | Eicosanoid | 14,15-diHETrE | 350.8 | 0.63 |
| 2 | Phospholipid | LPC 20:5 | 353.6 | 0.63 |
| 3 | Phospholipid | LPC 0-18:0 | 354.0 | 0.61 |
| 4 | Phospholipid | PE 38:0, PE O-40:7 | 354.3 | 0.60 |
| 5 | Phospholipid | PC 36:5 | 354.6 | 0.61 |
| 6 | Phospholipid | PC 34:4 | 355.9 | 0.59 |
| 7 | Phospholipid | PC 40:8, PC O-40:1 | 356.2 | 0.60 |
| 8 | Phospholipid | PC O-34:4 | 357.2 | 0.58 |
| 9 | Eicosanoid | 11,12-diHETrE | 358.0 | 0.59 |
| 10 | Phospholipid | PC 40:7, PC O-40:0 | 358.2 | 0.57 |
| 11 | Phospholipid | PC 42:9, PC O-42:2 | 358.3 | 0.58 |
| 12 | Sphingomyelin | SM 34:1 | 358.7 | 0.61 |
| 13 | Phospholipid | LPC O-16:0 | 358.7 | 0.57 |
| 14 | Phospholipid | LPC 18:2 | 358.9 | 0.55 |
| 15 | Eicosanoid | 16 HDoHE | 359.0 | 0.55 |
| 16 | Phospholipid | PC 42:8, PC O-42:1 | 359.2 | 0.58 |
| 17 | Phospholipid | LPC 18:1 | 359.4 | 0.56 |
| 18 | Sphingomyelin | SM 34:0 | 359.4 | 0.60 |
| 19 | Phospholipid | PC 38:6 | 359.6 | 0.56 |
| 20 | Phospholipid | PC 42:4 | 359.6 | 0.56 |
| 21 | Sterol | Desmosterol | 359.7 | 0.56 |
| 22 | Phospholipid | PE 32:1 | 359.8 | 0.59 |
| 23 | Phospholipid | LPC 22:0 | 359.9 | 0.57 |
| 24 | Phospholipid | LPC 18:0 | 359.9 | 0.56 |
| 25 | Phospholipid | PC 38:5 | 360.0 | 0.56 |
| 26 | Phospholipid | PE 34:1 | 360.1 | 0.58 |
| 27 | Sphingomyelin | SM 36:2 | 360.3 | 0.56 |
| 28 | Phospholipid | PE 34:0 | 360.4 | 0.58 |
| 29 | Phospholipid | LPC 22:5 | 360.4 | 0.57 |
| 30 | Phospholipid | LPC 20:3 | 360.4 | 0.56 |
| 31 | Phospholipid | PC 42:5 | 360.4 | 0.54 |
| 32 | Phospholipid | PC 34:3 | 360.5 | 0.56 |
| 33 | Sphingomyelin | SM 36:3 | 360.5 | 0.54 |
| 34 | Phospholipid | LPC 22:6 | 360.6 | 0.56 |
| 35 | Phospholipid | PE 34:2 | 360.7 | 0.57 |
| 36 | Phospholipid | PE 32:0 | 360.9 | 0.57 |
| 37 | Phospholipid | PC 32:0 | 361.0 | 0.56 |
| 38 | Phospholipid | PC 42:7, PC O-42:0 | 361.0 | 0.56 |
| 39 | Phospholipid | PC 42:10, PC O-42:3 | 361.1 | 0.56 |
| 40 | Phospholipid | PC 36:4 | 361.2 | 0.56 |
| 41 | Eicosanoid | 11-HETE | 361.4 | 0.52 |
| 42 | Phospholipid | LPC 16:0 | 361.4 | 0.57 |
| 43 | Phospholipid | PC O-36:0 | 361.5 | 0.53 |
| 44 | Phospholipid | PC O-34:0 | 361.6 | 0.56 |
| 45 | Phospholipid | PE 36:1 | 361.6 | 0.54 |
| 46 | Eicosanoid | 8-HETE | 361.6 | 0.57 |
| 47 | Phospholipid | PC O-40:4 | 361.7 | 0.54 |
| 48 | Phospholipid | PI 34:1 | 361.8 | 0.58 |
| 49 | Ceramide | CER d18:0/24:1 | 361.8 | 0.54 |
| 50 | Eicosanoid | 19,20 DiHDPA | 362.0 | 0.54 |
| 51 | Phospholipid | LPC 16:1 | 362.1 | 0.55 |
| 52 | Phospholipid | PE 36:3 | 362.1 | 0.54 |
| 53 | Phospholipid | LPC 20:4 | 362.1 | 0.55 |
| 54 | Phospholipid | PC 38:4 | 362.3 | 0.54 |
| 55 | Sphingomyelin | SM 36:4 | 362.3 | 0.51 |
| 56 | Phospholipid | PC O-36:1 | 362.3 | 0.55 |
| 57 | Sterol | 7,27-dihydroxy-cholesterol | 362.3 | 0.54 |
| 58 | Sterol | 25-hydroxy-cholesterol | 362.4 | 0.54 |
| 59 | Phospholipid | PE 36:2 | 362.4 | 0.53 |
| 60 | Phospholipid | PC 38:0, PC O-40:7 | 362.4 | 0.53 |
| 61 | Phospholipid | PC O-38:2 | 362.5 | 0.56 |
| 62 | Phospholipid | PI 38:5 | 362.6 | 0.52 |
| 63 | Sphingomyelin | SM 38:2 | 362.7 | 0.52 |
| 64 | Sphingomyelin | SM 34:4 | 362.7 | 0.53 |
| 65 | Sphingomyelin | SM 32:0 | 362.7 | 0.56 |
| 66 | Phospholipid | PC 40:6 | 362.7 | 0.53 |
| 67 | Eicosanoid | 12,13 EpOME | 362.8 | 0.57 |
| 68 | Phospholipid | PE 42:10 | 362.9 | 0.56 |
| 69 | Ceramide | CER d18:0/16:0 | 362.9 | 0.55 |
| 70 | Ceramide | CER d18:0/24:0 | 362.9 | 0.51 |
| 71 | Phospholipid | PE 38:1, PE O-40:8 | 362.9 | 0.54 |
| 72 | Phospholipid | LPE 20:4 | 362.9 | 0.54 |
| 73 | Phospholipid | PC 42:6 | 362.9 | 0.53 |
| 74 | Eicosanoid | 20cooh AA | 363.0 | 0.54 |
| 75 | Eicosanoid | 13-HODE | 363.0 | 0.52 |
| 76 | Phospholipid | PC O-38:3 | 363.0 | 0.53 |
| 77 | Sphingomyelin | SM 32:2 | 363.0 | 0.53 |
| 78 | Phospholipid | PE 40:7, PE O-40:0 | 363.0 | 0.53 |
| 79 | Ceramide | CER d18:1/22:6 | 363.1 | 0.54 |
| 80 | Ceramide | CER P-d18:1/20:3 | 363.1 | 0.52 |
| 81 | Phospholipid | PC O-34:2 | 363.1 | 0.54 |
| 82 | Ceramide | CER d18:1/18:1 | 363.1 | 0.51 |
| 83 | Phospholipid | PC 40:2 | 363.1 | 0.54 |
| 84 | Sphingomyelin | SM 34:3 | 363.1 | 0.52 |
| 85 | Ceramide | CER d18:1/18:0 | 363.1 | 0.53 |
| 86 | Phospholipid | PC O-42:11 | 363.2 | 0.53 |
| 87 | Phospholipid | PC 40:3 | 363.2 | 0.53 |
| 88 | Phospholipid | PC O-36:5 | 363.2 | 0.53 |
| 89 | Phospholipid | PE 36:5 | 363.2 | 0.51 |
| 90 | Sterol | 27-hydroxy-cholesterol | 363.2 | 0.52 |
| 91 | Ceramide | CER P-d18:0/18:0 | 363.2 | 0.53 |
| 92 | Sterol | Cholestanol | 363.2 | 0.55 |
| 93 | Ceramide | CER P-d18:1/24:0 | 363.2 | 0.52 |
| 94 | Phospholipid | PC O-40:3 | 363.2 | 0.52 |
| 95 | Phospholipid | PC O-36:2 | 363.2 | 0.54 |
| 96 | Ceramide | CER P-d18:1/20:5 | 363.2 | 0.54 |
| 97 | Eicosanoid | 5-HETE | 363.2 | 0.49 |
| 98 | Phospholipid | PC 38:3 | 363.3 | 0.53 |
| 99 | Phospholipid | PC O-40:5 | 363.3 | 0.52 |
| 100 | Phospholipid | PI 36:1 | 363.3 | 0.53 |
| 101 | Eicosanoid | 9-HODE | 363.3 | 0.53 |
| 102 | Ceramide | CER d18:1/16:0 | 363.3 | 0.53 |
| 103 | Phospholipid | PE 38:6 | 363.3 | 0.51 |
| 104 | Phospholipid | PE 40:6 | 363.3 | 0.52 |
| 105 | Phospholipid | PC 32:2 | 363.3 | 0.54 |
| 106 | Phospholipid | PI 34:2 | 363.3 | 0.55 |
| 107 | Phospholipid | LPC 22:4 | 363.4 | 0.53 |
| 108 | Phospholipid | PE 40:8, PE O-40:1 | 363.4 | 0.53 |
| 109 | Sphingomyelin | SM 32:1 | 363.4 | 0.55 |
| 110 | Phospholipid | PE 40:4 | 363.4 | 0.52 |
| 111 | Ceramide | CER d18:1/20:0 | 363.4 | 0.52 |
| 112 | Phospholipid | PC 36:2 | 363.4 | 0.52 |
| 113 | Ceramide | CER d18:1/26:0 | 363.4 | 0.53 |
| 114 | Phospholipid | LPE 18:0 | 363.4 | 0.53 |
| 115 | Phospholipid | PC 40:5 | 363.4 | 0.52 |
| 116 | Phospholipid | PC O-36:6 | 363.4 | 0.52 |
| 117 | Ceramide | CER d18:1/22:5 | 363.4 | 0.52 |
| 118 | Phospholipid | PC 36:0, PC O-38:7 | 363.4 | 0.51 |
| 119 | Sterol | Lanosterol | 363.5 | 0.52 |
| 120 | Ceramide | CER d18:1/20:1 | 363.5 | 0.54 |
| 121 | Phospholipid | PS 38:3 | 363.5 | 0.57 |
| 122 | Phospholipid | PC 32:1 | 363.5 | 0.54 |
| 123 | Eicosanoid | tetranor 12-HETE | 363.5 | 0.52 |
| 124 | Phospholipid | PE 30:1 | 363.5 | 0.54 |
| 125 | Phospholipid | PC O-42:10 | 363.5 | 0.53 |
| 126 | Phospholipid | PC O-40:6 | 363.5 | 0.52 |
| 127 | Phospholipid | LPC 22:3 | 363.5 | 0.51 |
| 128 | Eicosanoid | 12,13 diHOME | 363.5 | 0.50 |
| 129 | Phospholipid | PI 36:2 | 363.5 | 0.53 |
| 130 | Phospholipid | PE 36:0, PE O-38:7 | 363.5 | 0.52 |
| 131 | Phospholipid | PE 32:2 | 363.5 | 0.52 |
| 132 | Phospholipid | PE 34:3 | 363.5 | 0.54 |
| 133 | Eicosanoid | 9-oxoODE | 363.6 | 0.52 |
| 134 | Ceramide | CER P-d18:1/18:1 | 363.6 | 0.52 |
| 135 | Phospholipid | PI 36:3 | 363.6 | 0.53 |
| 136 | Phospholipid | PE 36:4 | 363.6 | 0.53 |
| 137 | Phospholipid | PI 36:4 | 363.6 | 0.51 |
| 138 | Phospholipid | PS 36:1 | 363.6 | 0.49 |
| 139 | Sterol | Campesterol | 363.6 | 0.53 |
| 140 | Ceramide | CER P-d18:0/22:1 | 363.6 | 0.51 |
| 141 | Sterol | 7a-hydroxy-4-cholesten-3-one | 363.6 | 0.51 |
| 142 | Ceramide | CER P-d18:1/22:0 | 363.6 | 0.52 |
| 143 | Phospholipid | PI 38:4 | 363.6 | 0.52 |
| 144 | Phospholipid | PC 34:0, PC O-36:7 | 363.6 | 0.51 |
| 145 | Eicosanoid | 5,6-diHETrE | 363.6 | 0.47 |
| 146 | Sterol | 14-demethyl-lanosterol | 363.6 | 0.50 |
| 147 | Phospholipid | PC O-36:4 | 363.6 | 0.52 |
| 148 | Ceramide | CER d18:0/22:0 | 363.6 | 0.52 |
| 149 | Ceramide | CER d18:1/24:1 | 363.7 | 0.51 |
| 150 | Ceramide | CER P-d18:0/20:5 | 363.7 | 0.52 |
| 151 | Ceramide | CER d18:1/22:4 | 363.7 | 0.51 |
| 152 | Sphingomyelin | SM 36:1 | 363.7 | 0.51 |
| 153 | Phospholipid | PE 42:9 | 363.7 | 0.53 |
| 154 | Phospholipid | PE 38:5 | 363.7 | 0.51 |
| 155 | Phospholipid | PC 38:1, PC O-40:8 | 363.7 | 0.51 |
| 156 | Phospholipid | LPE 18:2 | 363.8 | 0.54 |
| 157 | Phospholipid | PC 34:1 | 363.8 | 0.51 |
| 158 | Phospholipid | PC 38:2, PC O-40:9 | 363.8 | 0.52 |
| 159 | Ceramide | CER P-d18:1/18:0 | 363.8 | 0.51 |
| 160 | Sterol | 24-hydroxy-cholesterol | 363.8 | 0.52 |
| 161 | Eicosanoid | 14 HDoHE | 363.8 | 0.48 |
| 162 | Ceramide | CER P-d18:0/16:0 | 363.8 | 0.49 |
| 163 | Phospholipid | PC O-42:5 | 363.8 | 0.52 |
| 164 | Phospholipid | PC O-34:1 | 363.8 | 0.52 |
| 165 | Phospholipid | PC 36:3 | 363.8 | 0.52 |
| 166 | Phospholipid | PS 36:2 | 363.8 | 0.49 |
| 167 | Phospholipid | PC 40:4 | 363.8 | 0.52 |
| 168 | Phospholipid | PE 38:7 | 363.8 | 0.52 |
| 169 | Eicosanoid | 12-HETE | 363.8 | 0.48 |
| 170 | Phospholipid | PS 40:5 | 363.8 | 0.56 |
| 171 | Sterol | 7-dehydrocholesterol | 363.8 | 0.53 |
| 172 | Phospholipid | PC O-42:9 | 363.8 | 0.52 |
| 173 | Phospholipid | PC O-38:6 | 363.8 | 0.52 |
| 174 | Phospholipid | PC 40:1 | 363.8 | 0.52 |
| 175 | Phospholipid | PE 42:7 | 363.8 | 0.51 |
| 176 | Phospholipid | PC O-36:3 | 363.8 | 0.52 |
| 177 | Sterol | 7a-hydroxy-cholesterol | 363.8 | 0.49 |
| 178 | Phospholipid | PI 38:3 | 363.8 | 0.49 |
| 179 | Phospholipid | PC 40:0 | 363.8 | 0.52 |
| 180 | Phospholipid | PC O-38:5 | 363.8 | 0.51 |
| 181 | Ceramide | CER d18:1/20:3 | 363.8 | 0.51 |
| 182 | Phospholipid | PS 38:4 | 363.8 | 0.53 |
| 183 | Sterol | Dihydro-lanosterol | 363.8 | 0.52 |
| 184 | Phospholipid | PE 40:5 | 363.8 | 0.50 |
| 185 | Ceramide | CER P-d18:0/18:1 | 363.8 | 0.53 |
| 186 | Phospholipid | LPE 16:0 | 363.8 | 0.51 |
| 187 | Phospholipid | PC 42:11, PC O-42:4 | 363.8 | 0.50 |
| 188 | Ceramide | CER P-d18:1/22:1 | 363.8 | 0.51 |
| 189 | Eicosanoid | 15-HETrE | 363.8 | 0.52 |
| 190 | Eicosanoid | 9,10 diHOME | 363.8 | 0.47 |
| 191 | Ceramide | CER P-d18:1/16:0 | 363.8 | 0.52 |
| 192 | Sterol | 4β-hydroxycholesterol | 363.9 | 0.49 |
| 193 | Ceramide | CER d18:1/20:4 | 363.9 | 0.52 |
| 194 | Ceramide | CER P-d18:0/16:1 | 363.9 | 0.51 |
| 195 | Ceramide | CER d18:1/22:0 | 363.9 | 0.52 |
| 196 | Eicosanoid | dhk PGD2 | 363.9 | 0.52 |
| 197 | Phospholipid | PE 38:4 | 363.9 | 0.49 |
| 198 | Eicosanoid | 15-HETE | 363.9 | 0.51 |
| 199 | Phospholipid | LPE 18:1 | 363.9 | 0.50 |
| 200 | Sterol | Sitosterol | 363.9 | 0.50 |
| 201 | Phospholipid | PC 36:1 | 363.9 | 0.50 |
| 202 | Sphingomyelin | SM 34:2 | 363.9 | 0.49 |
| 203 | Phospholipid | PE 38:3 | 363.9 | 0.49 |
| 204 | Eicosanoid | 9-HOTrE | 363.9 | 0.52 |
| 205 | Phospholipid | LPE 22:6 | 363.9 | 0.50 |
| 206 | Phospholipid | PE 42:8 | 363.9 | 0.48 |
| 207 | Phospholipid | PC 42:0 | 363.9 | 0.49 |
| 208 | Eicosanoid | 9,10 EpOME | 363.9 | 0.52 |
| 209 | Phospholipid | PE 38:2, PE O-40:9 | 363.9 | 0.51 |
| 210 | Phospholipid | PE 40:9, PE O-40:2 | 363.9 | 0.52 |
| 211 | Ceramide | CER d18:1/24:0 | 363.9 | 0.50 |
| 212 | Ceramide | CER P-d18:1/16:1 | 363.9 | 0.51 |
| 213 | Ceramide | CER P-d18:1/20:4 | 363.9 | 0.48 |
| 214 | Ceramide | CER P-d18:1/24:1 | 363.9 | 0.51 |
| 215 | Ceramide | CER d18:1/22:1 | 363.9 | 0.49 |
| 216 | Phospholipid | PC 34:2 | 363.9 | 0.5 |
| 217 | Ceramide | CER d18:1/26:1 | 363.9 | 0.51 |
| 218 | Phospholipid | PC O-34:3 | 363.9 | 0.53 |

*The binary outcome (NASH vs. NAFL) was regressed on each lipid using simple logistic regression.

TABLE 9
Top 9 lipids with the lowest BIC from the 1-lipid model*

| Rank | Class of lipid | Lipid | BIC |
|---|---|---|---|
| 1 | Eicosanoid | 14,15-diHETrE | 350.8 |
| 2 | Phospholipid | LPC 20:5 | 353.6 |
| 3 | Phospholipid | LPC O-18:0 | 354.0 |
| 4 | Phospholipid | PE 38:0, PE O-40:7 | 354.3 |
| 5 | Phospholipid | PC 36:5 | 354.6 |
| 6 | Phospholipid | PC 34:4 | 355.9 |
| 7 | Phospholipid | PC 40:8, PC O-40:1 | 356.2 |
| 8 | Phospholipid | PC O-34:4 | 357.2 |
| 9 | Eicosanoid | 11,12-diHETrE | 358.0 |

*BIC was calculated from logistic regression with the NASH status (NASH vs. NAFL) as an outcome and each lipid as a covariate. The lower BIC is, the higher the model provides information.

TABLE 10
Selected final model*^{†‡} (N = 304)

| | OR | Standardized OR^{§} | p |
|---|---|---|---|
| 14,15-diHETrE (pmol/mL) | 4.17 (1.73, 10.1) | 1.69 (1.22, 2.34) | 0.001 |
| Lyso-phosphatidylcholine O-18:0 (NI/mL) | 0.89 (0.83, 0.95) | 0.53 (0.37, 0.77) | <0.001 |
| Phosphatidylcholine 34:4 (NI/mL) | 0.95 (0.92, 0.99) | 0.70 (0.54, 0.91) | 0.007 |

NI/mL = normalized intensity relative to an internal standard/mL; OR = odds ratio.
*Among the best 9 lipids with the lowest BIC in the 1-lipid model, all the combinations of these lipids, a total of 2⁹ = 512 combinations, were compared for BIC using multiple regression models in which multiple lipids were included as covariates. The model that yielded the lowest BIC was selected as the final model.
^{†}Twenty best models are shown in Table B.
^{‡}The equation is:

[symbol]

[symbol]

[symbol]

^{§}OR associated with an increase of lipid by 1 SD. [symbol]
indicates data missing or illegible when filed

The best model had an AUROC of 0.67 (95 confidence interval (CI)=0.61, 0.72) and a sensitivity of 26 (20, 32) at 90 specificity (Table 11). The positive and negative predictive values (PPV and NPV) were 20 0 and 91 at 10 NASH prevalence.

TABLE 11
Diagnostic performance of the final model. Leave-one-out cross-
validated AUROC and other performance indicators (N = 372)

| | AUROC | Sensitivity (%) | Specificity (%) | Cutoff |
|---|---|---|---|---|
| | (95% CI) | (95% CI) | (95% CI) | probability |
| At 90% | 0.67 | 26 | — | 0.851 |
| specificity^{†} | (0.61, 0.72) | (20, 32) | | |
| At 90% | | — | 36 | 0.591 |
| sensitivity^{†} | | | (25, 47) | |
| At maximum | | 84 | 44 | 0.637 |
| Youden's | | (78, 88) | (33, 56) | |
| index | | | | |

^{†}Sensitivity and specificity fixed at ≥90% and closest to 90%.

Numerous modifications and variations in the invention as set forth in the above illustrative examples are expected to occur to those skilled in the art. Consequently only such limitations as appear in the appended claims should be placed on the invention.

What is claimed:

1. A method of identifying nonalcoholic fatty liver disease (NAFLD) in a subject, comprising:
(a) obtaining a biological sample from the subject;
(b) measuring the level of a plurality of bioactive lipids selected from the group consisting of at least dhk-PGD2, 5-HETE and ceramide P-d18: 1/20:5, and optionally one or more additional compounds selected from the group consisting of CER P-d18: 1/18:0, SM 36:3, LPE 18:1, LPC 0-18:0, SM 34:3, PC 42:10, PC O—, LPC 18:2, PC 42:9, PC 0-42:2, and PC 40:0; and
(c) comparing the levels of dhk-PGD2, 5-HETE and ceramide P-d18:1/20:5 in the biological sample obtained from the subject to a control sample, wherein a difference in the levels is indicative of NAFLD.

2. The method of claim 1, comprising measuring at least dhk-PGD2, 5-HETE, ceramide P-d18: 1/20:5 and LPE 18:1.

3. The method of claim 1, comprising measuring at least dhk-PGD2, 5-HETE, ceramide P-d18: 1/20:5, LPE 18:1 and SM 34:3.

4. The method of claim 1, comprising measuring at least dhk-PGD2, 5-HETE, ceramide P-d18:1/20:5, LPE 18:1, SM 34:3 and PC 43:9, PC O-42:2.

5. The method of claim 1, comprising measuring at least dhk-PGD2, 5-HETE, ceramide P-d18: 1/20:5, ceramide P-d18: 1/18:0, and LPE 18:1.

6. The method of claim 1, comprising measuring at least dhk-PGD2, 5-HETE, ceramide P-d18:1/20:5, ceramide P-d18: 1/18:0, LPE 18:1, and SM 36:3.

7. The method of claim 1, comprising measuring at least dhk-PGD2, 5-HETE, ceramide P-d18: 1/20:5, ceramide P-d18: 1/18:0, LPE 18:1, SM 36:3, and LPC O 18:0.

8. The method of claim 1, comprising measuring at least dhk-PGD2, 5-HETE, ceramide P-d18: 1/20:5, ceramide P-d18:1/18:0, LPE 18:1, SM 36:3, LPC O 18:0, and LPC 18:2.

9. The method of claim 1, further comprising determining the area under receiver operating characteristic curve (AUROC) based upon a ratio of the levels of the bioactive lipids matched with deuterated internal standards of the same bioactive lipids.

10. The method of claim 1, wherein the biological sample is selected from the group consisting of blood, blood plasma and blood serum.

11. The method of claim 1, wherein the plurality of bioactive lipids are measured by liquid chromatography mass spectrometry.

12. The method of claim 1, wherein the plurality of bioactive lipids are measured by gas chromatography mass spectrometry.

13. The method of claim 1, further comprising determining whether a subject with NAFLD has NASH by measuring a second set of bioactive lipids, selected from the group consisting of at least 14,15-diHETrE, LPC 0-18:0 and PC 34:4, and optionally one or more additional compounds selected from the group consisting of LPC 20:5, PE 38:0, PE 0-40:7, PC 36:5, PC 40:8, PC 0-40:1 and PC 0-34:4 and wherein if there is a difference in the second set of bioactive lipids compared to a control or a control-NALFD level the levels are indicative of NASH.

14. A method of identifying nonalcoholic steatohepatitis (NASH) in a subject, comprising:
(a) obtaining a biological sample from the subject;
(b) measuring the level of a plurality of bioactive lipids selected from the group consisting of at least 14, 15-diHETrE, LPC O-18:0 and PC 34:4, and optionally one or more additional compounds selected from the group consisting of LPC 20:5, PE 38:0, PE 0-40:7, PC 36:5, PC 40:8, PC 0-40:1 and PC 0-34:4; and
(c) comparing the levels of at least 14, 15-diHETrE, LPC 0-18:0 and PC 34:4 in the biological sample obtained from the subject to a control sample, wherein a difference in the levels is indicative of NASH.

15. The method of claim 14, comprising measuring at least 14,15-diHETrE, LPC 0-18:0, PC 34:4 and PE 38:0, PE 0-40:7.

16. The method of claim 14, comprising measuring at least 14,15-diHETrE, LPC 0-18:0, PC 34:4, and LPC 20:5.

17. The method of claim 14, comprising measuring at least at least 14, 15-diHETrE, LPC 0-18:0, PC 34:4, and PC 36:5.

18. The method of claim 14, comprising measuring at least at least 14,15-diHETrE, LPC 0-18:0, PC 34:4, and PC 40:8, PC 0-40:1.

19. The method of claim 14, further comprising determining the area under receiver operating characteristic curve (AUROC) based upon a ratio of the levels of the bioactive lipids matched with deuterated internal standards of the same bioactive lipids.

20. The method of claim 14, wherein the biological sample is selected from the group consisting of blood, blood plasma and blood serum.

21. The method of claim 14, wherein the plurality of bioactive lipids are measured by liquid chromatography mass spectrometry.

22. The method of claim 14, wherein the plurality of bioactive lipids are measured by gas chromatography mass spectrometry.
